(19) United States
(12) Patent Application Publication
(10) Pub. No.: US 2022/0333091 A1
(43) Pub. Date: Oct. 20, 2022

(54) RECOMBINANT BOTULINUM NEUROTOXIN WITH IMPROVED SAFETY MARGIN AND REDUCED IMMUNOGENICITY

(71) Applicants: NEW YORK UNIVERSITY, New York, NY (US); CYTODEL, INC., New York, NY (US)

(72) Inventors: Konstantin ICHTCHENKO, New York, NY (US); Philip A. BAND, West Orange, NJ (US)

(21) Appl. No.: 17/765,016

(22) PCT Filed: Sep. 30, 2020
(86) PCT No.: PCT/US2020/053583

§ 371 Date: Mar. 30, 2022

Related U.S. Application Data
(60) Provisional application No. 62/908,365, filed on Sep. 30, 2019.

(51) Int. Cl. C12N 9/52

(52) U.S. Cl. CPC C12N 9/52; C12Y 304/24069; A61K 38/00

(57) ABSTRACT

Described herein are recombinant Clostridium botulinum neurotoxins comprising a light chain of a Clostridium botulinum neurotoxin, wherein the light chain comprises a mutation that causes minimal structural interference to the light chain protease; a heavy chain of a Clostridium botulinum neurotoxin, wherein the light and heavy chains are linked by a disulfide bond. The recombinant Clostridium botulinum neurotoxin has a 2-20 fold reduced toxicity compared to wild type Clostridium botulinum neurotoxin. Also described is a treatment method.

This application claims priority benefit of U.S. Provisional Patent Application Ser. No. 62/908,365, filed Sep. 30, 2019, which is hereby incorporated by reference in its entirety.

### FIELD

The Present application relates to a recombinant botulinum neurotoxin with improved safety margin and reduced immunogenicity.

### BACKGROUND

The Clostridial neurotoxins are a family of structurally similar proteins that target the neuronal machinery for synaptic vesicle exocytosis. Produced by anaerobic bacteria of the Clostridium genus, botulinum neurotoxins (“BoNT”s, seven immunologically distinct subtypes, A-G) and Tetanus neurotoxin (“TeNT”) are the most poisonous substances known on a per-weight basis, with an LD₅₀ in the range of 0.5-2.5 ng/kg when administered by intravenous or intramuscular routes (National Institute of Occupational Safety and Health, “Registry of Toxic Effects of Chemical Substances (R-TECS),” Cincinnati, Ohio: National Institute of Occupational Safety and Health (1996)). BoNTs target cholinergic nerves at their neuromuscular junction, inhibiting acetylcholine release and causing peripheral neuromuscular blockade (Simpson, “Identification of the Major Steps in botulinum Toxin Action,” Annu. Rev. Pharmacol. Toxicol. 44:167-193 (2004)).

A genetic engineering platform that enables rational design of therapeutic agents based on Clostridial toxin genes was described in U.S. Pat. No. 7,785,606 to Ichtchenko and Band. The genetic engineering scheme was based on a two-step approach. Gene constructs, expression systems, and purification schemes were designed that produce physiologically active, recombinant Clostridial neurotoxin derivatives. The recombinant toxin derivatives retained structural features important for developing therapeutic candidates, or useful biologic reagents. Using the genetic constructs and expression systems developed by this paradigm, selective point mutations were then introduced to create recombinant atoxic Clostridial neurotoxin derivatives.

Treatment methods that involve contacting a patient with isolated, physiologically active, toxic, Clostridial neurotoxin derivatives have been described in U.S. Pat. No. 7,785,606 to Band and Ichtchenko. Also, isolated, physiologically active, toxic and atoxic Clostridium botulinum neurotoxin derivatives that have an S6 peptide sequence fused to the N-terminus of the proteins to enable site-specific attachment of cargo using Sfp phosphopantetheinyl transferase have been described as suitable for treatment (U.S. Patent Application Publication No. 2011/0206616 to Ichtchenko and Band). Likewise, methods that involve treatment with an atoxic derivative of a Clostridial neurotoxin lacking a cargo attachment sequence at its N-terminus, and having a much higher LD₅₀ than a toxic derivative of a Clostridial neurotoxin or a wild type Clostridial neurotoxin have been described (U.S. Pat. No. 9,345,549 to Vasquez-Cintron, Ichtchenko, and Band). However, such reduced toxicity derivatives can cause an unwanted immunogenic response in certain situations, such as during treatment of dystonia of large muscle groups requiring doses of BoNT that can result in off-target adverse events, and non-responsiveness to repeat treatments.

The present application is directed to overcoming this and other limitations in the art.

### SUMMARY

Described herein is a recombinant Clostridium botulinum neurotoxin comprising a light chain of a Clostridium botulinum neurotoxin, where the light chain comprises a mutation corresponding to Y₃₆₆>X of BoNT A, where X is a amino acid that causes minimal structural intereference to the light chain protease; a heavy chain of a Clostridium botulinum neurotoxin, where the light and heavy chains are linked by a disulfide bond. The recombinant Clostridium botulinum neurotoxin has a 2-20 fold reduced toxicity compared to wild type Clostridium botulinum neurotoxin.

Further described herein is a recombinant Clostridium botulinum neurotoxin comprising a light chain of a Clostridium botulinum neurotoxin, where the light chain comprises a mutation corresponding to E₂₂₄>X of BoNT A, where X is a amino acid that causes minimal structural intereference to the light chain protease; a heavy chain of a Clostridium botulinum neurotoxin, where the light and heavy chains are linked by a disulfide bond. The recombinant Clostridium botulinum neurotoxin has a 2-20 fold reduced toxicity compared to wild type Clostridium botulinum neurotoxin.

Also described is a treatment method. This method involves selecting a subject in need of therapeutic treatment involving induction of muscle paralysis and contacting the subject with a recombinant Clostridium botulinum neurotoxin disclosed herein to induce muscle paralysis in the subject to treat the subject, with the proviso that the neurotoxin derivative does not possess a cargo attachment peptide sequence at its N-terminus.

The Clostridium botulinum neurotoxins described herein provide an increased safety margin while at the same time providing a decreased risk of immunogenic response as compared to other atoxic derivatives.

Genetic constructs and expression systems described herein are shown to produce a family of recombinant BoNT derivatives, with conformational and trafficking properties similar to the wild type BoNT toxins. The Clostridium botulinum neurotoxins described herein provide an increased safety margin while at the same time providing a decreased risk of immunogenic response as compared to other atoxic derivatives. Thus, Clostridium botulinum neurotoxins can be produced in reduced toxicicity forms, having a potency comparable to that of the wild type toxin but with an improved safety margin. The LD₅₀ of the neurotoxins described herein is slightly higher than that of the wild type toxin.

Neurotoxin derivatives with reduced toxicity (see U.S. Pat. No. 7,785,606 to Ichtchenko et al., which is hereby incorporated by reference in its entirety) were unexpectedly shown to have in vivo activity similar to the wild type neurotoxins used for pharmaceutical purposes, except these molecules at higher doses induced immunogenic responses in animals administered the higher doses. Neurotoxin derivatives described in U.S. Pat. No. 9,345,549 offer significant treatment benefits over current pharmaceutical preparations of wild type neurotoxins produced from cultures. In particular, the reduced toxicity derivatives described in U.S. Pat. No. 9,345,549 are safer, providing distinct advantages for medical uses and production/ manufacturing. The improved therapeutic index will enable application to conditions where the toxicity of wild type neurotoxins limit their use because of safety concerns. The neurotoxins described herein offer surprising and significant additional treatment benefits in that they exhibit a decreased risk of immunogenic response as compared to other atoxic neurotoxin derivatives.

### BRIEF DESCRIPTION OF THE DRAWINGS

FIGS. 1A-B are a comparative alignment of amino acid sequences of representative subtypes of seven wild type botulinum neurotoxin serotypes, including Clostridium botulinum serotype A1 (wt BoNT A) (SEQ ID NO:1), Clostridium botulinum serotype B1 (wt BoNT B) (SEQ ID NO:2), Clostridium botulinum serotype C1 (wt BoNT C) (SEQ ID NO:3), Clostridium botulinum serotype D (wt BoNT D) (SEQ ID NO:4), Clostridium botulinum serotype E1 (wt BoNT E) (SEQ ID NO:5), Clostridium botulinum serotype F1 (wt BoNT F) (SEQ ID NO:6), and Clostridium botulinum serotype G (wt BoNT G) (SEQ ID NO:7). Gaps have been introduced to maximize homology. Amino acids identical in ≥50% of compared sequences are shown in black boxes. Amino acids constituting the active site of the catalytic domain of metalloprotease are marked by stars. Disulfide bridge between neurotoxin cysteine residues of the light and heavy chain are shown as a long horizontal bracket. The amino acid residues constituting the minimal catalytic domain of the light chain are grey highlighted. The first amino acid of the protein heavy chain, is shown with a white arrow (and labeled Heavy Chain), as is the first amino acid considered to constitute the receptor-binding domain (labeled Receptor Binding Domain). Amino acids absent in the mature dichain BoNT A molecule along with the aligned amino acids of the other BoNT serotypes are boxed. A white arrow is also positioned at the first amino acid of the neurotoxins' light chain.

FIG. 2 is a photograph showing the results of in vivo studies performed by intramuscular injection into the lateral gastrocnemius with 0.5 μg/mouse of BoNT A/ad-0 (experimental) in 3 μl of 0.9% NaCl or by injecting 3 μl of 0.9% of NaCl without BoNT A/ad-0 (control). Muscle paralysis and digital abduction was recorded 48 hours after. The two upper panel photographs show control mice, with the arrow in the upper right photograph indicating the site of injection. The three lower panel photographs show experimental mice. Digital abduction muscle paralysis was only observed in mice injected with BoNT A/ad-0.Experimental, n=10. Control, n=5. Representative results are shown in the photographs in the three bottom panels.

FIG. 3 depicts Cyto-014 production. Lanes 1-12: SDS-PAGE analysis of Cyto-014 purification by tandem Ni-NTA and StrepTactin affinity chromatography. Lanes 13-16: SDSPAGE (reducing and non-reducing) confirming TEV protease activation of Cyto-014. Lane 2—clarified Cyto-014-containing media (Ni-NTA loading material); 3—Ni-NTA flow through; 4-6—sequential washes with low imidazole; 7-250 mM imidazole eluate; 8-11—StrepTactin flow through and washes; 12-5 mMD-desthiobiotin eluate (purified Cyto-014); 13,14-2.5 μm and 1 μg activated Cyto-014, non-reduced; 15,16-2.5 μg and 1 μg activated Cyto-014, reduced (˜150-kD uncleaned.

FIG. 4 demonstrates that Cyto-014 cleaves SNAP-25 as efficiently as wild type BoNT A1 in vitro per Murine Lethality Assay Unit added to neuronal cultures (see Pearce et al., “Measurement of botulinum Toxin Activity: Evaluation of the Lethality Assay,” Toxicol Appl Pharmacol. 128:69-77 (1994), which is hereby incorporated by reference in its entirety). Primary fetal rat neurons were cultured as described previously and treated with Cyto-014 (lanes 1-4) or wt BoNT A1 (lanes 5-8). Each molecule showed similar extent of SNAP25 cleavage when normalized to LD₅₀ units.

FIG. 5 demonstrates that Cyto-014 IP-LD₅₀ is ˜8-fold less toxic than wt BoNT A1. The dose-response for IP toxicity was determined by injecting Cyto-014 (closed circles) or wt BoNT A1 (open circles), into the intraperitoneal space of CD-1 mice (n=5 per dose group) and assessing their survival over 5 days. The Cyto-014 IP-LD₅₀ was determined to be 35 pg for a 25-g mouse, 8-fold less toxic than the IP-LD₅₀ of 4.6 pg for wt BoNT A1.

FIG. 6 demonstrates that Cyto-014 IM-LD₅₀ is ˜7-fold higher than IP-LD₅₀. The dose-response for IM toxicity was determined by injecting Cyto-014 (closed circles) or wt BoNT A1 (open circles) into the lateral gastrocnemius muscle of CD-1 mice (n=5 to 10 per dose group) and assessing their survival over 5 days. The Cyto-014 IMLD₅₀ was determined to be 240 pg for a 25-g mouse.

FIG. 7 demonstrates that Cyto-014 IM ED₅₀ is ˜16-fold higher than wt BoNT/1. The dose-response for DAS scores was determined by injecting Cyto-014 (closed circles) or wt BoNT A1 (open circles) into the lateral gastrocnemius muscle of CD-1 mice (n=5 to 10 per dose group) and assessing their ipsilateral DAS over 9 days. The Cyto-014 IM-ED₅₀ was determined from the peak DAS of each mouse during monitoring, and calculated to be 12 pg for a 25-g mouse.

FIGS. 8A-8D depict results of a pooled sera ELISA experiment described in Example 3.

FIGS. 9A-9B depict the results of an individual sera ELISA experiment described in Example 3.

FIG. 10 depicts a F-140 monoclonal antibody standard curve used in the ELISA experiments described in Example 3.

FIGS. 11A-11B depict the results of the mouse protection assay described in Example 3.

### DETAILED DESCRIPTION OF THE INVENTION

Described herein is a recombinant Clostridium botulinum neurotoxin comprising a light chain of a Clostridium botulinum neurotoxin, where the light chain comprises a mutation corresponding to Y₃₆₆>X of BoNT A, where X is an amino acid that causes minimal structural interference to the light chain protease; a heavy chain of a Clostridium botulinum neurotoxin, where the light and heavy chains are linked by a disulfide bond. The recombinant Clostridium botulinum neurotoxin has a 2-20 fold reduced toxicity compared to wild type Clostridium botulinum neurotoxin.

Further described herein is a recombinant Clostridium botulinum neurotoxin comprising a light chain of a Clostridium botulinum neurotoxin, where the light chain comprises a mutation corresponding to E₂₂₄>X of BoNT A, where X is a amino acid that causes minimal structural intereference to the light chain protease; a heavy chain of a Clostridium botulinum neurotoxin, where the light and heavy chains are linked by a disulfide bond. The recombinant Clostridium botulinum neurotoxin has a 2-20 fold reduced toxicity compared to wild type Clostridium botulinum neurotoxin.

Also described is a treatment method. This method involves selecting a subject in need of therapeutic treatment involving induction of muscle paralysis and contacting the subject with a recombinant Clostridium botulinum neurotoxin disclosed herein to induce muscle paralysis in the subject to treat the subject, with the proviso that the neurotoxin derivative does not possess a cargo attachment peptide sequence at its N-terminus.

According to one embodiment, the Clostridium botulinum neurotoxin is a derivative of a Clostridium botulinum neurotoxin. Clostridium botulinum has multiple serotypes, including BoNT A-G (SEQ ID NOs:1-7), BoNT/H (GenBank Accession No. KG015617, which is hereby incorporated by reference in its entirety), and BoNT/X (GenBank Accession No. WP_045538952, and Masuyer et al., “Structural Characterisation of the Catalytic Domain of botulinum Neurotoxin X—High Activity and Unique Substrate Specificity,” Nature 8:4518 (2017), which are each hereby incorporated by reference in their entirety) as well as numerous subtypes. See also Peck et al., “Historical Perspectives and Guidelines for botulinum Neurotoxin Subtype Nomenclature,” Toxins 9:38 (2017); Pirazzini et al., “botulinum Neurotoxins: Biology, Pharmacology, and Toxicology,” Pharmacological Reviews 69:200-235 (2017), which are hereby incorporated by reference in their entirety. Suitable derivatives of a Clostridium botulinum neurotoxin may be derivatives of any of the Clostridium botulinum serotypes and subtypes. In addition, suitable Clostridium botulinum neurotoxin of the present application may be derivatives of more than one Clostridium botulinum serotype and subtype. For example, it may be desirable to have a derivative of a Clostridium botulinum neurotoxin constructed of a light chain (LC) region from one Clostridium botulinum serotype (e.g., serotype A, BoNT A1) and a heavy chain (HC) region from another Clostridium botulinum serotype (e.g., serotype B, BoNT B1). Also, suitable Clostridium botulinum neurotoxins of the present application include chimeras using other receptor ligands, e.g., epidermal growth factor (“EGF”) for LC delivery to cancer cells (see U.S. Patent Application Publication no. 2012/0064059 to Foster et al., which is hereby incorporated by reference in its entirety).

An example of Clostridium botulinum serotype A1 (wild type BoNT A1) has an amino acid sequence as set forth in GenBank Accession No. CAL82360, which is hereby incorporated by reference in its entirety (SEQ ID NO:1):

MPFVNKQFNY KDPVNGVDIA YIKIPNAGQM QPVKAFKIHN
KIWVIPERDT FTNPEEGDLN PPPEAKQVPV SYYDSTYLST
DNEKDNYLKG VTKLFERIYS TDLGRMLLTS IVRGIPFWGG
STIDTELKVI DTNCINVIQP DGSYRSEELN LVIIGPSADI
IQFECKSFGH EVLNLTRNGY GSTQYIRFSP DFTFGFEESL
EVDTNPLLGA GKFATDPAVT LAHELIHAGH RLYGIAINPN
RVFKVNTNAY YEMSGLEVSF EELRTFGGHD AKFIDSLQEN
EFRLYYYNKF KDIASTLNKA KSIVGTTASL QYMKNVFKEK
YLLSEDTSGK FSVDKLKFDK LYKMLTEIYT EDNFVKFFKV
LNRKTYLNFD KAVFKINIVP KVNYTIYDGF NLRNTNLAAN
FNGQNTEINN MNFTKLKNFT GLFEFYKLLC VRGIITSKTK
SLDKGYNKAL NDLCIKVNNW DLFFSPSEDN FTNDLNKGEE
ITSDTNIEAA EENISLDLIQ QYYLTFNFDN EPENISIENL
SSDIIGQLEL MPNIERFPNG KKYELDKYTM FHYLRAQEFE
HGKSRIALTN SVNEALLNPS RVYTFFSSDY VKKVNKATEA
AMFLGWVEQL VYDFTDETSE VSTTDKIADI TIIIPYIGPA
LNIGNMLYKD DFVGALIFSG AVILLEFIPE IAIPVLGTFA
LVSYIANKVL TVQTIDNALS KRNEKWDEVY KYIVTNWLAK
VNTQIDLIRK KMKEALENQA EATKAIINYQ YNQYTEEEKN
NINFNIDDLS SKLNESINKA MININKFLNQ CSVSYLMNSM
IPYGVKRLED FDASLKDALL KYIYDNRGTL IGQVDRLKDK
VNNTLSTDIP FQLSKYVDNQ RLLSTFTEYI KNIINTSILN
LRYESNHLID LSRYASKINI GSKVNFDPID KNQIQLFNLE
SSKIEVILKN AIVYNSMYEN FSTSFWIRIP KYFNSISLNN
EYTIINCMEN NSGWKVSLNY GEIIWTLQDT QEIKQRVVFK
YSQMINISDY INRWIFVTIT NNRLNNSKIY INGRLIDQKP
ISNLGNIHAS NNIMFKLDGC RDTHRYIWIK YFNLFDKELN
EKEIKDLYDN QSNSGILKDF WGDYLQYDKP YYMLNLYDPN
KYVDVNNVGI RGYMYLKGPR GSVMTTNIYL NSSLYRGTKF
IIKKYASGNK DNIVRNNDRV YINVVVKNKE YRLATNASQA
GVEKILSALE IPDVGNLSQV VVMKSKNDQG ITNKCKMNLQ
DNNGNDIGFI GFHQFNNIAK LVASNWYNRQ IERSSRTLGC
SWEFIPVDDG WGERPL

An example of Clostridium botulinum serotype B1 (wild type BoNT B1) has an amino acid sequence as set forth in GenBank Accession No. ACA46990, which is hereby incorporated by reference in its entirety (SEQ ID NO:2):

MPVTINNFNY NDPIDNNNII MMEPPFARGT GRYYKAFKIT
DRIWIIPERY TFGYKPEDFN KSSGIFNRDV CEYYDPDYLN
TNDKKNIFLQ TMIKLFNRIK SKPLGEKLLE MIINGIPYLG
DRRVPLEEFN TNIASVTVNK LISNPGEVER KKGIFANLII
FGPGPVLNEN ETIDIGIQNH FASREGFGGI MQMKFCPEYV
SVFNNVQENK GASIFNRRGY FSDPALILMH ELIHVLHGLY
GIKVDDLPIV PNEKKFFMQS TDAIQAEELY TFGGQDPSII
TPSTDKSIYD KVLQNFRGIV DRLNKVLVCI SDPNININIY
KNKFKDKYKF VEDSEGKYSI DVESFDKLYK SLMFGFTETN
IAENYKIKTR ASYFSDSLPP VKIKNLLDNE IYTIEEGFNI
SDKDMEKEYR GQNKAINKQA YEEISKEHLA VYKIQMCKSV
KAPGICIDVD NEDLFFIADK NSFSDDLSKN ERIEYNTQSN
YIENDFPINE LILDTDLISK IELPSENTES LTDFNVDVPV
YEKQPAIKKI FTDENTIFQY LYSQTFPLDI RDISLTSSFD
DALLFSNKVY SFFSMDYIKT ANKVVEAGLF AGWVKQIVND
FVIEANKSNT MDKIADISLI VPYIGLALNV GNETAKGNFE
NAFEIAGASI LLEFIPELLI PVVGAFLLES YIDNKNKIIK
TIDNALTKRN EKWSDMYGLI VAQWLSTVNT QFYTIKEGMY
KALNYQAQAL EEIIKYRYNI YSEKEKSNIN IDFNDINSKL
NEGINQAIDN INNFINGCSV SYLMKKMIPL AVEKLLDFDN
TLKKNLLNYI DENKLYLIGS AEYEKSKVNK YLKTIMPFDL
SIYTNDTILI EMFNKYNSEI LNNIILNLRY KDNNLIDLSG
YGAKVEVYDG VELNDKNQFK LTSSANSKIR VTQNQNIIFN
SVFLDFSVSF WIRIPKYKND GIQNYIHNEY TIINCMKNNS
GWKISIRGNR IIWTLIDING KTKSVFFEYN IREDISEYIN
RWFFVTITNN LNNAKIYING KLESNTDIKD IREVIANGEI
IFKLDGDIDR TQFIWMKYFS IFNTELSQSN IEERYKIQSY
SEYLKDFWGN PLMYNKEYYM FNAGNKNSYI KLKKDSPVGE
ILTRSKYNQN SKYINYRDLY IGEKFIIRRK SNSQSINDDI
VRKEDYIYLD FFNLNQEWRV YTYKYFKKEE EKLFLAPISD
SDEFYNTIQI KEYDEQPTYS CQLLFKKDEE STDEIGLIGI
HRFYESGIVF EEYKDYFCIS KWYLKEVKRK PYNLKLGCNW
QFIPKDEGWT E

An example of Clostridium botulinum serotype C1 (wild type BoNT C) has an amino acid sequence as set forth in (SEQ ID NO:3):

MPITINNFNY SDPVDNKNIL YLDTHLNTLA NEPEKAFRIT
GNIWVIPDRF SRNSNPNLNK PPRVTSPKSG YYDPNYLSTD
SDKDPFLKEI IKLFKRINSR EIGEELIYRL STDIPFPGNN
NTPINTFDFD VDFNSVDVKT RQGNNWVKTG SINPSVIITG
PRENIIDPET STFKLTNNTF AAQEGFGALS IISISPRFML
TYSNATNDVG EGRFSKSEFC MDPILILMHE LNHAMHNLYG
IAIPNDQTIS SVTSNIFYSQ YNVKLEYAEI YAFGGPTIDL
IPKSARKYFE EKALDYYRSI AKRLNSITTA NPSSFNKYIG
EYKQKLIRKY RFVVESSGEV TVNRNKFVEL YNELTQIFTE
FNYAKIYNVQ NRKIYLSNVY TPVTANILDD NVYDIQNGFN
IPKSNLNVLF MGQNLSRNPA LRKVNPENML YLFTKFCHKA
IDGRSLYNKT LDCRELLVKN TDLPFIGDIS DVKTDIFLRK
DINEETEVIY YPDNVSVDQV ILSKNTSEHG QLDLLYPSID
SESEILPGEN QVFYDNRTQN VDYLNSYYYL ESQKLSDNVE
DFTFTRSIEE ALDNSAKVYT YFPTLANKVN AGVQGGLFLM
WANDVVEDFT TNILRKDTLD KISDVSAIIP YIGPALNISN
SVRRGNFTEA FAVTGVTILL EAFPEFTIPA LGAFVIYSKV
QERNEIIKTI DNCLEQRIKR WKDSYEWMMG TWLSRIITQF
NNISYQMYDS LNYQAGAIKA KIDLEYKKYS GSDKENIKSQ
VENLKNSLDV KISEAMNNIN KFIRECSVTY LFKNMLPKVI
DELNEFDRNT KAKLINLIDS HNIILVGEVD KLKAKVNNSF
QNTIPFNIFS YTNNSLLKDI INEYFNNIND SKILSLQNRK
NTLVDTSGYN AEVSEEGDVQ LNPIFPFDFK LGSSGEDRGK
VIVTQNENIV YNSMYESFSI SFWIRINKWV SNLPGYTIID
SVKNNSGWSI GIISNFLVFT LKQNEDSEQS INFSYDISNN
APGYNKWFFV TVTNNMMGNM KIYINGKLID TIKVKELTGI
NFSKTITFEI NKIPDTGLIT SDSDNINMWI RDFYIFAKEL
DGKDINILFN SLQYTNVVKD YWGNDLRYNK EYYMVNIDYL
NRYMYANSRQ IVFNTRRNNN DFNEGYKIII KRIRGNTNDT
RVRGGDILYF DMTINNKAYN LFMKNETMYA DNHSTEDIYA
IGLREQTKDI NDNIIFQIQP MNNTYYYASQ IFKSNFNGENV
ISGICSIGTY RFRLGGDWYR HNYLVPTVKQ GNYASLLEST
STHWGFVPVS E

An example of Clostridium botulinum serotype D (wild type BoNT D) has an amino acid sequence as set forth in (SEQ ID NO:4):

MTWPVKDFNY SDPVNDNDIL YLRIPQNKLI TTPVKAFMIT
QNIWVIPERF SSDTNPSLSK PPRPTSKYQS YYDPSYLSTD
EQKDTFLKGI IKLFKRINER DIGKKLINYL VVGSPFMGDS
STPEDTFDFT RHTTNIAVEK FENGSWKVTN IITPSVLIFG
PLPNILDYTA SLTLQGQQSN PSFEGFGTLS ILKVAPEFLL
TFSDVTSNQS SAVLGKSIFC MDPVIALMHE LTHSLHQLYG
INIPSDKRIR PQVSEGFFSQ DGPNVQFEEL YTFGGLDVEI
IPQIERSQLR EKALGHYKDI AKRLNNINKT IPSSWISNID
KYKKIFSEKY NFDKDNTGNF VVNIDKFNSL YSDLTNVMSE
VVYSSQYNVK NRTHYFSRHY LPVFANILDD NIYTIRDGFN
LTNKGFNIEN SGQNIERNPA LQKLSSESVV DLFTKVCLRL
TKNSRDDSTC IKVKNNRLPY VADKDSISQE IFENKIITDE
TNVQNYSDNF SLDESILDGQ VPINPEIVDP LLPNVNMEPL
NLPGEEIVFY DDITKYVDYL NSYYYLESQK LSNNVENITL
TTSVEEALGY SNKIYTFLPS LAEKVNKGVQ AGLFLNWANE
VVEDFTTNIM KKDTLDKISD VSVIIPYIGP ALNIGNSALR
GNFKQAFATA GVAFLLEGFP EFTIPALGVF TFYSSIQERE
KIIKTIENCL EQRVKRWKDS YQWMVSNWLS RITTQFNHIN
YQMYDSLSYQ ADAIKAKIDL EYKKYSGSDK ENIKSQVENL
KNSLDVKISE AMNNINKFIR ECSVTYLFKN MLPKVIDELN
KFDLRTKTEL INLIDSHNII LVGEVDRLKA KVNESFENTM
PFNIFSYTNN SLLKDIINEY FNSINDSKIL SLQNKKNALV
DTSGYNAEVR VGDNVQLNTI YTNDFKLSSS GDKIIVNLNN
NILYSAIYEN SSVSFWIKIS KDLTNSHNEY TIINSIEQNS
GWKLCIRNGN IEWILQDVNR KYKSLIFDYS ESLSHTGYTN
KWFFVTITNN IMGYMKLYIN GELKQSQKIE DLDEVKLDKT
IVFGIDENID ENQMLWIRDF NIFSKELSNE DINIVYEGQI
LRNVIKDYWG NPLKFDTEYY IINDNYIDRY IAPESNVLVL
VRYPDRSKLY TGNPITIKSV SDKNPYSRIL NGDNIILHML
YNSRKYMIIR DTDTIYATQG GECSQNCVYA LKLQSNLGNY
GIGIFSIKNI VSKNKYCSQI FSSFRENTML LADIYKPWRF
SFKNAYTPVA VTNYETKLLS TSSFWKFISR DPGWVE

An example of Clostridium botulinum serotype E1 (wild type BoNT E) has an amino acid sequence as set forth in (SEQ ID NO:5):

MPKINSFNYN DPVNDRTILY IKPGGCQEFY KSFNIMKNIW
IIPERNVIGT TPQDFHPPTS LKNGDSSYYD PNYLQSDEEK
DRFLKIVTKI FNRINNNLSG GILLEELSKA NPYLGNDNTP
DNQFHIGDAS AVEIKFSNGS QDILLPNVII MGAEPDLFET
NSSNISLRNN YMPSNHGFGS IAIVTFSPEY SFRFNDNSMN
EFIQDPALTL MHELIHSLHG LYGAKGITTK YTITQKQNPL
ITNIRGTNIE EFLTFGGTDL NIITSAQSND IYTNLLADYK
KIASKLSKVQ VSNPLLNPYK DVFEAKYGLD KDASGIYSVN
INKFNDIFKK LYSFTEFDLA TKFQVKCRQT YIGQYKYFKL
SNLLNDSIYN ISEGYNINNL KVNFRGQNAN LNPRIITPIT
GRGLVKKIIR FCKNIVSVKG IRKSICIEIN NGELFFVASE
NSYNDDNINT PKEIDDTVTS NNNYENDLDQ VILNFNSESA
PGLSDEKLNL TIQNDAYIPK YDSNGTSDIE QHDVNELNVF
FYLDAQKVPE GENNVNLTSS IDTALLEQPK IYTFFSSEFI
NNVNKPVQAA LFVSWIQQVL VDFTTEANQK STVDKIADIS
IVVPYIGLAL NIGNEAQKGN FKDALELLGA GILLEFEPEL
LIPTILVFTI KSFLGSSDNK NKVIKAINNA LKERDEKWKE
VYSFIVSNWM TKINTQFNKR KEQMYQALQN QVNAIKTIIE
SKYNSYTLEE KNELTNKYDI KQIENELNQK VSIAMNNIDR
FLTESSISYL MKLINEVKIN KLREYDENVK TYLLNYIIQH
GSILGESQQE LNSMVTDTLN NSIPFKLSSY TDDKILISYF
NKFFKRIKSS SVLNMRYKND KYVDTSGYDS NININGDVYK
YPTNKNQFGI YNDKLSEVNI SQNDYIIYDN KYKNFSISFW
VRIPNYDNKI VNVNNEYTII NCMRDNNSGW KVSLNHNEII
WTLEDNAGIN QKLAFNYGNA NGISDYINKW IFVTITNDRL
GDSKLYINGN LIDQKSILNL GNIHVSDNIL FKIVNCSYTR
YIGIRYFNIF DKELDETEIQ TLYSNEPNTN ILKDFWGNYL
LYDKEYYLLN VLKPNNFIDR RKDSTLSINN IRSTILLANR
LYSGIKVKIQ RVNNSSTNDN LVRKNDQVYI NFVASKTHLF
PLYADTATTN KEKTIKISSS GNRFNQVVVM NSVGNNTMNF
KNNNGNNIGL LGFKADTVVA STWYYTHMRD HTNSNGCFWN
FISEEHGWQE K

An example of Clostridium botulinum serotype F1 (wild type BoNT F) has an amino acid sequence as set forth in (SEQ ID NO:6):

MPVVINSFNY NDPVNDDTIL YMQIPYEEKS KKYYKAFEIM
RNVWIIPERN TIGTDPSDFD PPASLENGSS AYYDPNYLTT
DAEKDRYLKT TIKLFKRINS NPAGEVLLQE ISYAKPYLGN
EHTPINEFHP VTRTTSVNIK SSTNVKSSII LNLLVLGAGP
DIFENSSYPV RKLMDSGGVY DPSNDGFGSI NIVTFSPEYE
YTFNDISGGY NSSTESFIAD PAISLAHELI HALHGLYGAR
GVTYKETIKV KQAPLMIAIK PIRLEEFLTF GGQDLNIITS
AMKEKIYNNL LANYEKIATR LSRVNSAPPE YDINEYKDYF
QWKYGLDKNA DGSYTVNENK FNEIYKKLYS FTEIDLANKF
KVKCRNTYFI KYGFLKVPNL LDDDIYTVSE GFNIGNLAVN
NRGQNIKLNP KIIDSIPDKG LVEKIVKFCK SVIPRKGTKA
PPRLCIRVNN RELFFVASES SYNENDINTP KEIDDTTNLN
NNYRNNLDEV ILDYNSETIP QISNQTLNTL VQDDSYVPRY
DSNGTSEIEE HNVVDLNVFF YLHAQKVPEG ETNISLTSSI
DTALSEESQV YTFFSSEFIN TINKPVHAAL FISWINQVIR
DFTTEATQKS TFDKIADISL VVPYVGLALN IGNEVQKENF
KEAFELLGAG ILLEFVPELL IPTILVFTIK SFIGSSENKN
KIIKAINNSL MERETKWKEI YSWIVSNWLT RINTQFNKRK
EQMYQALQNQ VDAIKTVIEY KYNNYTSDER NRLESEYNIN
NIREELNKKV SLAMENIERF ITESSIFYLM KLINEAKVSK
LREYDEGVKE YLLDYISEHR SILGNSVQEL NDLVTSTLNN
SIPFELSSYT NDKILILYFN KLYKKIKDNS ILDMRYENNK
FIDISGYGSN ISINGDVYIY STNRNQFGIY SSKPSEVNIA
QNNDIIYNGR YQNFSISFWV RIPKYFNKVN LNNEYTIIDC
IRNNNSGWKI SLNYNKIIWT LQDTAGNNQK LVFNYTQMIS
ISDYINKWIF VTITNNRLGN SRIYINGNLI DEKSISNLGD
IHVSDNILFK IVGCNDTRYV GIRYFKVFDT ELGKTEIETL
YSDEPDPSIL KDFWGNYLLY NKRYYLLNLL RTDKSITQNS
NFLNINQQRG VYQKPNIFSN TRLYTGVEVI IRKNGSTDIS
NTDNFVRKND LAYINVVDRD VEYRLYADIS IAKPEKIIKL
IRTSNSNNSL GQIIVMDSIG NNTMNFQNN NGGNIGLLGF
HSNNLVASSW YYNNIRKNTS SNGCFWSFIS KEHGWQEN

An example of Clostridium botulinum serotype G (wild type BoNT G) has an amino acid sequence as set forth in GenBank Accession No. KIE44899, which is hereby incorporated by reference in its entirety) (SEQ ID NO:7):

MPVNIKNFNY NDPINNDDII MMEPFNDPGP GTYYKAFRII
DRIWIVPERF TYGFQPDQFN ASTGVFSKDV YEYYDPTYLK
TDAEKDKFLK TMIKLFNRIN SKPSGQRLLD MIVDAIPYLG
NASTPPDKFA ANVANVSINK KIIQPGAEDQ IKGLMTNLII
FGPGPVLSDN FTDSMIMNGH SPISEGFGAR MMIRFCPSCL
NVFNNVQENK DTSIFSRRAY FADPALTLMH ELIHVLHGLY
GIKISNLPIT PNTKEFFMQH SDPVQAEELY TFGGHDPSVI
SPSTDMNIYN KALQNFQDIA NRLNIVSSAQ GSGIDISLYK
QIYKNKYDFV EDPNGKYSVD KDKFDKLYKA LMFGFTETNL
AGEYGIKTRY SYFSEYLPPI KTEKLLDNTI YTQNEGFNIA
SKNLKTEFNG QNKAVNKEAY EEISLEHLVI YRIAMCKPVM
YKNTGKSEQC IIVNNEDLFF IANKDSFSKD LAKAETIAYN
TQNNTIENNF SIDQLILDND LSSGIDLPNE NTEPFTNFDD
IDIPVYIKQS ALKKIFVDGD SLFEYLHAQT FPSNIENLQL
TNSLNDALRN NNKVYTFFST NLVEKANTVV GASLFVNWVK
GVIDDFTSES TQKSTIDKVS DVSIIIPYIG PALNVGNETA
KENFKNAFEI GGAAILMEFI PELIVPIVGF FTLESYVGNK
GHIIMTISNA LKKRDQKWTD MYGLIVSQWL STVNTQFYTI
KERMYNALNN QSQAIEKIIE DQYNRYSEED KMNINIDFND
IDFKLNQSIN LAINNIDDFI NQCSISYLMN RMIPLAVKKL
KDFDDNLKRD LLEYIDTNEL YLLDEVNILK SKVNRHLKDS
IPFDLSLYTK DTILIQVFNN YISNISSNAI LSLSYRGGRL
IDSSGYGATM NVGSDVIFND IGNGQFKLNN SENSNITAHQ
SKFVVYDSMF DNFSINFWVR TPKYNNNDIQ TYLQNEYTII
SCIKNDSGWK VSIKGNRIIW TLIDVNAKSK SIFFEYSIKD
NISDYINKWF SITITNDRLG NANIYINGSL KKSEKILNLD
RINSSNDIDF KLINCTDTTK FVWIKDFNIF GRELNATEVS
SLYWIQSSTN TLKDFWGNPL RYDTQYYLFN QGMQNIYIKY
FSKASMGETA PRTNFNNAAI NYQNLYLGLR FIIKKASNSR
NINNDNIVRE GDYIYLNIDN ISDESYRVYV LVNSKEIQTQ
LFLAPINDDP TFYDVLQIKK YYEKTTYNCQ ILCEKDTKTF
GLFGIGKFVK DYGYVWDTYD NYFCISQWYL RRISENINKL
RLGCNWQFIP VDEGWTE

By “derivative” it is meant that the Clostridium botulinum neurotoxin is substantially similar to the wild type toxin, but has been modified slightly as described herein. For example, derivatives include Clostridium botulinum neurotoxins that are at least 60%, 70%, 80%, 85%, 90%, 95%, 96%, 97%, 98%, or 99% identical to a wild type neurotoxin.

In some embodiments the derivative is a light chain derivative. In some embodiments, the light chain derivative is at least 60%, 70%, 80%, 85%, 90%, 95%, 96%, 97%, 98%, or 99% identical to the light chain of a wild type neurotoxin. In some embodiments the derivative is a heavy chain derivative. In some embodiments, the heavy chain derivative is at least 60%, 70%, 80%, 85%, 90%, 95%, 96%, 97%, 98%, or 99% identical to the heavy chain of a wild type neurotoxin.

Isolated derivatives of a Clostridium botulinum neurotoxin are physiologically active. This physiological activity includes, but is not limited to, toxin immunogenicity, trans-and intra-cellular trafficking, cell recognition and targeting, and paralytic activity. In one embodiment, Clostridium botulinum neurotoxin is a derivative of a full-length Clostridial neurotoxin. In one embodiment, Clostridium botulinum neurotoxin is a derivative of a light chain Clostridial neurotoxin. In another embodiment, Clostridium botulinum neurotoxin is a derivative of a heavy chain Clostridial neurotoxin.

The Clostridium botulinum neurotoxin described herein does not have an S6 peptide sequence fused to the N-terminus of the neurotoxin, as described in U.S. Patent Application Publication No. 2011/0206616 to Icthtchenko and Band, which is hereby incorporated by reference in its entirety.

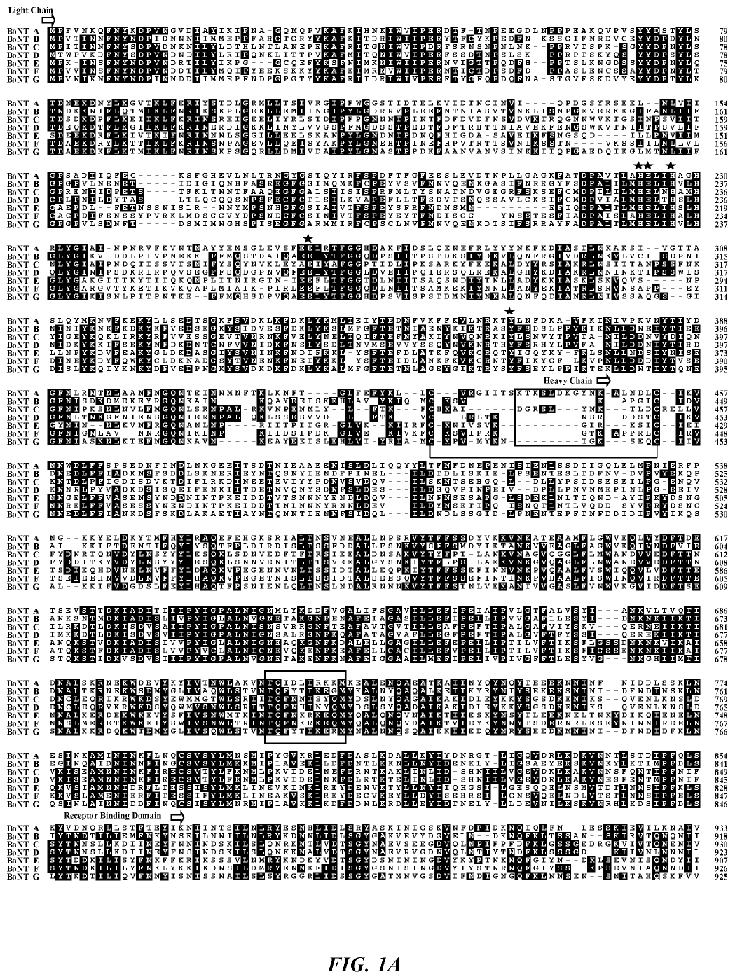

The mechanism of cellular binding and internalization of Clostridial neurotoxins is still not completely understood. The C-terminal portion of the heavy chain of all Clostridial neurotoxins binds to gangliosides (sialic acid-containing glycolipids), with a preference for gangliosides of the G_{1b} series (Montecucco et al., “Structure and Function of Tetanus and botulinum Neurotoxins,” Q. Rev. Biophys. 28:423-472 (1995); Montecucco, “How Do Tetanus and botulinum Toxins Bind to Neuronal Membranes?” TIBS 11:314-317 (1986); and Van Heyningen et al., “The Fixation of Tetanus Toxin by Ganglioside,” J. Gen. Microbiol. 24:107-119 (1961), which are hereby incorporated by reference in their entirety). The sequence responsible for ganglioside binding has been identified for the structurally similar TeNT molecule, and is located within the 34 C-terminal amino acid residues of its heavy chain. BoNT A, BoNT B, BoNT C, BoNT E, and BoNT F share a high degree of homology with TeNT in this region (FIGS. 1A-1B) (Shapiro et al., “Identification of a Ganglioside Recognition Domain of Tetanus Toxin Using a Novel Ganglioside Photoaffinity Ligand,” J. Biol. Chem. 272:30380-30386 (1997), which is hereby incorporated by reference in its entirety). Multiple types of evidence suggest the existence of at least one additional component involved in the binding of Clostridial neurotoxins to neuronal membranes (Montecucco et al., “Structure and Function of Tetanus and botulinum Neurotoxins,” Q. Rev. Biophys. 28:423-472 (1995); Montecucco, “How Do Tetanus and botulinum Toxins Bind to Neuronal Membranes?” TIBS 11:314-317 (1986), which are hereby incorporated by reference in their entirety). In two reports (Nishiki et al., “The High-Affinity Binding of Clostridium botulinum Type B Neurotoxin to Synaptotagmin II Associated with Gangliosides G_{T1b}/G_{D1a},” FEBS Len. 378:253-257 (1996); Dong et al., “Synaptotagmins I and II Mediate Entry of botulinum Neurotoxin B into Cells,” J. Cell Biol. 162:1293-1303 (2003), which are hereby incorporated by reference in their entirety), synaptotagmins were identified as possible candidates for the auxiliary BoNT B receptor, and synaptotagmins I and II were implicated as neuronal receptors for BoNT G (Rummel et al., “Synaptotagmins I and II Act as Nerve Cell Receptors for botulinum Neurotoxin G,” J. Biol. Chem. 279:30865-30870 (2004), which is hereby incorporated by reference in its entirety). Dong et al., “SV2 is the Protein Receptor for botulinum Neurotoxin A,” Science 312:592-596 (2006), which is hereby incorporated by reference in its entirety, showed that BoNT A enters neurons by binding to the synaptic vesicle protein SV2 (isoforms A, B, and C). However, despite the structural similarity in the putative receptor-binding domain of Clostridial neurotoxins, other toxin subtypes show no affinity for SV2 and instead may target synaptotagmins or synaptotagmin-related molecules. Lipid rafts (Herreros et al., “Lipid Rafts Act as Specialized Domains for Tetanus Toxin Binding and Internalization into Neurons,” Mol. Biol. Cell 12:2947-2960 (2001), which is hereby incorporated by reference in its entirety) have been implicated as a specialized domain involved in TeNT binding and internalization into neurons, but these domains are widely distributed on multiple cell types, and therefore cannot simply explain the high specificity of the toxins for neurons.

Clostridial neurotoxins are internalized through the presynaptic membrane by an energy-dependent mechanism (Montecucco et al., “Structure and Function of Tetanus and botulinum Neurotoxins,” Q. Rev. Biophys. 28:423-472 (1995); Matteoli et al., “Synaptic Vesicle Endocytosis Mediates the Entry of Tetanus Neurotoxin into Hippocampal Neurons,” Proc. Natl. Acad. Sci. USA 93:13310-13315 (1996); and Mukherjee et al., “Endocytosis,” Physiol. Rev. 77:759-803 (1997), which are hereby incorporated by reference in their entirety), and rapidly appear in vesicles where they are at least partially protected from degradation (Dolly et al., “Acceptors for botulinum Neurotoxin Reside on Motor Nerve Terminals and Mediate Its Internalization,” Nature 307:457-460 (1984); Critchley et al., “Fate of Tetanus Toxin Bound to the Surface of Primary Neurons in Culture: Evidence for Rapid Internalization,” J. Cell Biol. 100:1499-1507 (1985), which are hereby incorporated by reference in their entirety). The BoNT complex of light and heavy chains interacts with the endocytic vesicle membrane in a chaperone-like way, preventing aggregation and facilitating translocation of the light chain in a fashion similar to the protein conducting/translocating channels of smooth ER, mitochondria, and chloroplasts (Koriazova et al., “Translocation of botulinum Neurotoxin Light Chain Protease through the Heavy Chain Channel,” Nat. Struct. Biol. 10:13-18 (2003), which is hereby incorporated by reference in its entirety). Acidification of the endosome is believed to induce pore formation, which allows translocation of the light chain to the cytosol upon reduction of the interchain disulfide bond (Hoch et al., “Channels Formed by botulinum, Tetanus, and Diphtheria Toxins in Planar Lipid Bilayers: Relevance to Translocation of Proteins Across Membranes,” Proc. Natl. Acad. Sci. USA 82:1692-1696 (1985), which is hereby incorporated by reference in its entirety). Within the cytosol, the light chain displays a zinc-endopeptidase activity specific for protein components of the synaptic vesicle exocytosis apparatus. TeNT and BoNT B, BoNT D, BoNT F, and BoNT G recognize VAMP/synaptobrevin. This integral protein of the synaptic vesicle membrane is cleaved at a single peptide bond, which differs for each neurotoxin. BoNT A, BoNT C, and BoNT E recognize and cleave SNAP-25, a protein of the presynaptic membrane, at different sites within the carboxyl terminus segment. BoNT C also cleaves syntaxin, another protein of the nerve terminal plasmalemma (Montecucco et al., “Structure and Function of Tetanus and botulinum Neurotoxins,” Q. Rev. Biophys. 28:423-472 (1995); Sutton et al., “Crystal Structure of a SNARE Complex Involved in Synaptic Exocytosis at 2.4 \acute{Å} Resolution,” Nature 395:347-353 (1998), which are hereby incorporated by reference in their entirety). The cleavage of such components of the synaptic release machinery results in inhibition of acetylcholine release in motor neurons, ultimately leading to neuromuscular paralysis.

The isolated Clostridium botulinum neurotoxins employed in the methods of the present application are physiologically active. The endopeptidase activity responsible for Clostridial neurotoxin toxicity is believed to be associated with the presence of a HExxHxxH (SEQ ID NO:8) motif in the light chain, characteristic of metalloproteases (FIGS. 1A-1B). Mutagenesis of BoNT A light chain, followed by microinjection of the corresponding mRNA into presynaptic cholinergic neurons of Aplysia californica, allowed the minimal essential domain responsible for toxicity to be identified (Kurazono et al., “Minimal Essential Domains Specifying Toxicity of the Light Chains of Tetanus Toxin and botulinum Neurotoxin Type A,” J. Biol. Chem. 267:14721-14729 (1992), which is hereby incorporated by reference in its entirety). Site-directed mutagenesis of BoNT A light chain pinpointed the amino acid residues involved in Zn²⁺ coordination, and formation of the active metalloendoprotease core which cleaves SNAP-25 (Rigoni et al., “Site-Directed Mutagenesis Identifies Active-Site Residues of the Light Chain of botulinum Neurotoxin Type A,” Biochem. Biophys. Res. Commun. 288:1231-1237 (2001), which is hereby incorporated by reference in its entirety). The three-dimensional structures of Clostridial neurotoxins and their derivatives confirmed the mutagenesis results, and detailed the spatial organization of the protein domains. For the BoNT A holotoxin, crystal structure was obtained to a resolution of 3.3 \acute{Å} (Lacy et al., “Crystal Structure of botulinum Neurotoxin Type A and Implications for Toxicity,” Nat. Struct. Biol. 5:898-902 (1998), which is hereby incorporated by reference in its entirety). The BoNT B holotoxin crystal structure was determined at 1.8 and 2.6 A resolution (Swaminathan et al., “Structural Analysis of the Catalytic and Binding Sites of Clostridium botulinum Neurotoxin B,” Nat. Struct. Biol. 7:693-699 (2000), which is hereby incorporated by reference in its entirety). Recently, a crystal structure for BoNT E catalytic domain was determined to 2.1 \acute{Å} resolution (Agarwal et al., “Structural Analysis of botulinum Neurotoxin Type E Catalytic Domain and Its Mutant Glu212>Gln Reveals the Pivotal Role of the Glu212 Carboxylate in the Catalytic Pathway,” Biochemistry 43:6637-6644 (2004), which is hereby incorporated by reference in its entirety). The later study provided multiple interesting structural details, and helps explain the complete loss of metalloendoproteolytic activity in the BoNT E LC E₂₁₂>Q mutant. The availability of this detailed information on the relationship between the amino acid sequence and biological activities of Clostridial toxins enables the design of modified toxin genes with properties specifically altered for therapeutic goals.

Thus, in one embodiment, the Clostridium botulinum neurotoxin has a metalloprotease disabling mutation. Specific metalloprotease disabling mutations are described in U.S. Pat. No. 7,785,606 to Ichthchenko and Band, which is hereby incorporated by reference in its entirety. Additional point mutations can be introduced to further modify the characteristics of the neurotoxin, some of which are also described in U.S. Pat. No. 7,785,606 to Ichthchenko and Band, which is hereby incorporated by reference in its entirety. As used herein, “minimal structural interference” refers to substitutions that minimally alter the structure of the light chain protease active site in order to mantain hydrophobic interactions in the active center.

In one embodiment, the metalloprotease disabling mutation corresponds to Y₃₆₆>X of BoNT A, where X is an amino acid that causes minimal structural interference to the light chain protease. That is, the Clostridium botulinum neurotoxin comprises a mutation in a wild type BoNT molecule in which tyrosine is substituted for, e.g., phenylalanine at a position corresponding to amino acid 366 of BoNT A1 (SEQ ID NO:1), or corresponding to the starred Y position on the fifth row in the sequence alignment shown in FIG. 1A, which corresponds to Y₃₆₆ in BoNT A. The tyrosine at this position is highly conserved across all BoNT serotypes and subtypes. In other embodiments, the Clostridium botulinum neurotoxin comprises a mutation at the starred Y position on the fifth row of FIG. 1A of any Clostridium botulinum neurotoxin serotype or subtype. In some embodiments, the Clostridium botulinum neurotoxin is selected from BoNT serotype B1 (BoNT B) with a mutation of Y₃₇₃>X, BoNT serotype C1 (BoNT C) with a mutation of Y₃₇₅>X, BoNT serotype D (BoNT D) with a mutation of Y₃₇₅>X, BoNT serotype E1 (BoNT E) with a mutation of Y₃₅₁>X, BoNT serotype F1 (BoNT F) with a mutation of Y_{368>X}, BoNT serotype G (BoNT G) with a mutation of Y₃₇₂>X, BoNT serotype H (BoNT H) with a mutation of Y₃₆₆>X, and BoNT serotype X (BoNT X) with a mutation of Y₃₆₃>X. In some embodiments, there is only one metalloprotease disabling mutation in the Clostridium botulinum neurotoxin. In some embodiments, an amino acid change that causes minimal structural interference of the light chain protease (e.g., for Y₃₆₆>X in BoNT A or a corresponding mutation in another serotype) is a hydrophobic amino acid. In some embodiments, the only one metalloprotease disabling mutation that causes minimal structural interference of the light chain protease is a mutation corresponding to Y₃₆₆>F in BoNT A1. In some embodiments, the only one metalloprotease disabling mutation that causes minimal structural interference of the light chain protease is a mutation corresponding to Y₃₇₃>F in BoNT B1, Y₃₇₅>F in BoNT C1, Y₃₇₅>F in BoNT D, Y₃₅₁>F in BoNT E1, Y₃₆₈>F in BoNT F1, Y₃₇₂>F in BoNT G, Y₃₆₆>F in BoNT H, or Y₃₆₃>F in BoNT X.

In another embodiment, the metalloprotease disabling mutation corresponds to E₂₂₄>X of BoNT A, where X is an amino acid that causes minimal structural interference to the light chain protease. That is, the Clostridium botulinum neurotoxin comprises a mutation in a wild type BoNT molecule in which glutamate is substituted for, e.g., glutamine at a position corresponding to amino acid 224 of BoNT A1 (SEQ ID NO:1), or corresponding to the starred E position on the third row in the sequence alignment shown in FIG. 1A. The glutamate at this position is highly conserved across all BoNT serotypes and subtypes. In other embodiments, the Clostridium botulinum neurotoxin comprises a mutation at the starred E position in the third row of FIG. 1A of any Clostridium botulinum neurotoxin serotype or subtype. In some embodiments, the Clostridium botulinum neurotoxin is selected from BoNT serotype B1 (BoNT B) with a mutation of E₂₃₁>X, BoNT serotype C1 (BoNT C) with a mutation of E₂₃₀>X, BoNT serotype D (BoNT D) with a mutation of E₂₃₀>X, BoNT serotype E1 (BoNT E) with a mutation of E₂₁₃>X, BoNT serotype F1 (BoNT F) with a mutation of E₂₂₈>X, BoNT serotype G (BoNT G) with a mutation of E₂₃₁>X, BoNT serotype H (BoNT H) with a mutation of E₂₂₇>X, and BoNT serotype X (BoNT X) with a mutation of E₂₂₈>X. In some embodiments, there is only one metalloprotease disabiling mutation in the Clostridium botulinum neurotoxin. In some embodiments, an amino acid change that causes minimal structural interference of the light chain protease (e.g., for E₂₂₄>X in BoNT A or a corresponding mutation in another serotype) is a negatively charged amino acid or a structurally similar amino acid with an uncharged side chain where the E₂₂₄ (or corresponding position in another BoNT serotype) carboxyl-group is substituted with an amide (as in glutamine), but retains the same length aliphatic side chain as in glutamic acid. In some embodiments, the only one metalloprotease disabling mutation that causes minimal structural interference of the light chain protease is a mutation corresponding to E₂₂₄>Q in BoNT A1. In some embodiments, the only one metalloprotease disabling mutation that causes minimal structural interference of the light chain protease is a mutation corresponding to E₂₃₁>Q in BoNT B1, E₂₃₀>Q in BoNT C1, E₂₃₀>Q in BoNT D, E₂₁₃>Q in BoNT E1, E₂₂₈>Q in BoNT F1, E₂₃₁>Q in BoNT G, E₂₂₇>Q in BoNT H, or E₂₂₈>Q in BoNT X.

In another embodiment, metalloprotease disabling mutations corresponding to positions Y₃₆₆>X or E₂₂₄^{>}X of BoNT A can be made in other BoNT serotypes and sequences such as representative BoNT sequences shown in GenBank Accession Nos: CAL82360, CAA51824, ACA57525, ACQ51417, ACG50065, ACW83608, AFV13854, AJA05787, ACA46990, BAC22064, ABM73977, ABM73987, ACQ51206, BAF91946, AFD33678, AFN61309, BAA14235, BAA08418, EES90380, ABP48747, CAA43999, EF028404, ABM73980, BAC05434, AB037704, CAM91125, AER11391, AER11392, AFV91339, KF861920, KF861879, KF929215, ABS41202, CAA73972, ADA79575, GU213221, GU213212, AAA23263, ADK48765, AUCZ00000000, KIE44899, CFSAN024410, KG015617, and WP 045538952, each of which is hereby incorporated by reference in its entirety.

In one embodiment, the Clostridium botulinum neurotoxin has an LD₅₀ that is at least 5; 6; 7; 8; 9; 10; 11; 12; 13; 14; 15; 16; 17; 18; 19; 20; 30; 40; 50; 60; 70; 80; 90; 100; 200; 300; 400; 500; 600; 700; 800; or 900-fold higher than the LD₅₀ of wild type Clostridium botulinum neurotoxin. In some embodiments, the Clostridium botulinum neurotoxin has an LD₅₀ that is at least 5-fold, 10-fold, or 15-fold higher than the LD₅₀ of a wild type Clostridial neurotoxin. The particular mode of administration may affect the LD₅₀ of the derivative of a Clostridial neurotoxin.

In one embodiment, the Clostridium botulinum neurotoxin of the present application is produced by cleaving a propeptide. The propeptide is cleaved at the highly specific protease cleavage site to form a light and heavy chain, with molecular weights of approximately 50 kD and 100 kD, respectively. The light and heavy chain regions are linked by a disulfide bond.

In one embodiment, the propeptide is contacted with a highly specific protease (e.g., enterokinase or TEV protease) under conditions effective to enable cleavage at the intermediate region of the propeptide of the present application. The intermediate region is indicated by a box in FIG. 1A. Preferably, the expressed propeptide has one or more disulfide bridges. Disulfide bridges are indicated by a long horizontal bracket in FIG. 1A.

As discussed infra, Clostridial neurotoxins and their derivatives described herein are synthesized as single chain propeptides which are later activated by a specific proteolysis cleavage event, generating a dimer joined by a disulfide bond. These structural features can be illustrated using BoNT A as an example (numbered according to SEQ ID NO:1), and are generally applicable to all Clostridium botulinum serotypes. The mature BoNT A is composed of three functional domains of Mr ˜50,000, where the catalytic function responsible for toxicity is confined to the light chain (residues 1-437), the translocation activity is associated with the N-terminal half of the heavy chain (residues 448-872), and cell binding is associated with its C-terminal half (residues 873-1,296) (Johnson, “Clostridial Toxins as Therapeutic Agents: Benefits of Nature's Most Toxic Proteins,” Annu. Rev. Microbiol. 53:551-575 (1999); Montecucco et al., “Structure and Function of Tetanus and botulinum Neurotoxins,” Q. Rev. Biophys. 28:423-472 (1995), which are hereby incorporated by reference in their entirety).

Optimized expression and recovery of recombinant neurotoxins for BoNT serotypes in a native and physiologically active state is achieved by the introduction of one or more alterations to the nucleotide sequences encoding the BoNT propeptides, as discussed infra. This codon optimization is designed to maximize yield of recombinant derivatives of a Clostridial neurotoxin in the expression host, while retaining the native toxins' structure and biological activity.

Common structural features of the wild-type Clostridium botulinum neurotoxin propeptides are shown in FIGS. 1A-B. These structural features are illustrated using wt BoNT A propeptide (SEQ ID NO:1) as an example, and are generalized among all Clostridium botulinum serotypes. wt BoNT A propeptide has two chains, a light chain (“LC”) of Mr ˜50,000 and a heavy chain (“HC”) of Mr ˜100,000, linked by a disulfide bond between Cys₄₃₀ and Cys₄₅₄. As illustrated in FIGS. 1A-B, all seven BoNT serotype propeptides have a light chain region and a heavy chain region linked by a disulfide bond. Two essential Cys residues, one adjacent to the C-terminus of the light chain, and a second adjacent to the N-terminus of the heavy chain are present in all seven BoNT serotypes. These two Cys residues form the single disulfide bond holding the HC and LC polypeptides together in the mature neurotoxin. This disulfide bond enables the mature neurotoxin to accomplish its native physiological activities by permitting the HC and LC to carry out their respective biological roles in concert. The disulfide bond between HC and LC polypeptides in all seven serotypes is illustrated in FIG. 1A by the long horizontal bracket joining the involved Cys residues. The outlined box in FIG. 1A on row 6 illustrates the intermediate region defined by amino acid residues Lys₄₃₈-Lys₄₄₈ of wt BoNT A (SEQ ID NO:1). This intermediate region identifies the amino acids eliminated during maturation of wt BoNT A, and believed to be excised by a protease endogenous to the host microorganism. This cleavage event, described infra, generates the biologically active BoNT HC-LC dimer. The outlined amino acid residues in FIG. 1A, representing amino acid residues approximately in the 420 to 450 range for all seven BoNT serotypes, can be considered as a region “non-essential” to the toxins' physiological activity and, therefore, represent targets for directed mutagenesis in all seven BoNT serotypes.

All seven wt BoNT serotypes referred to herein contain Lys or Arg residues in the intermediate region defined by the box on row 6 of FIG. 1A, which make the propeptides susceptible to activation by trypsin. Native BoNT A propeptide recovered from young bacterial cultures can be activated by trypsinolysis, with production of intact, S-S bound light and heavy chain. Though multiple additional trypsin-susceptible sites are present in the native propeptides, they are resistant to proteolysis due to their spatial positions within the native toxin molecule (Dekleva et al., “Nicking of Single Chain Clostridium botulinum Type A Neurotoxin by an Endogenous Protease,” Biochem. Biophys. Res. Commun. 162:767-772 (1989); Lacy et al., “Crystal Structure of botulinum Neurotoxin Type A and Implications for Toxicity,” Nat. Struct. Biol. 5:898-902 (1998), which are hereby incorporated by reference in their entirety). A second site in the native propeptide of several BoNT serotypes can be susceptible to trypsin cleavage when subjected to higher enzyme concentrations or incubation times (Chaddock et al., “Expression and Purification of Catalytically Active, Non-Toxic Endopeptidase Derivatives of Clostridium botulinum Toxin Type A,” Protein Expr. Purif. 25:219-228 (2002), which is hereby incorporated by reference in its entirety). This native propeptide trypsin-susceptible site is located in the region adjacent to the toxin receptor binding domain. This region of the HC peptide is found to be exposed to solvent in BoNT serotypes for which information is available on their 3-D crystal structure (Lacy et al., “Crystal Structure of botulinum Neurotoxin Type A and Implications for Toxicity,” Nat. Struct. Biol. 5:898-902 (1998); Swaminathan et al., “Structural Analysis of the Catalytic and Binding Sites of Clostridium botulinum Neurotoxin B,” Nat. Struct. Biol. 7:693-699 (2000), which are hereby incorporated by reference in their entirety).

In one embodiment, the propeptide has an intermediate region connecting the light and heavy chain regions which has a highly specific protease cleavage site and no low-specificity protease cleavage sites. For purposes of the present application, a highly specific protease cleavage site has three or more specific adjacent amino acid residues that are recognized by the highly specific protease in order to permit cleavage (e.g., an enterokinase cleavage site or a TEV recognition sequence). In contrast, a low-specificity protease cleavage site has two or less adjacent amino acid residues that are recognized by a protease in order to enable cleavage (e.g., a trypsin cleavage site).

In all seven BoNT serotypes, the amino acid preceding the N-terminus of the heavy chain is a Lys or Arg residue which is susceptible to proteolysis with trypsin. This trypsin-susceptible site can be replaced with a five amino acid enterokinase cleavage site (i.e., DDDDK (SEQ ID NO:9)) upstream of the heavy chain's N-terminus. Alternatively, the trypsin-susceptible site can be replaced with a tobacco etch virus protease recognition (“TEV”) (i.e. ENLYFQ (SEQ ID NO:10), which cleaves with best efficiency if the next amino acid is G or S. Use of a TEV sequence enables a one-step heterodimer-forming cleavage event. See U.S. Patent Application Publication No. 2011/0206616 to Ichtchenko et al., which is hereby incorporated by reference in its entirety. Either of these modifications enables standardization activation with specific enzymes. Other highly specific proteases can be used. In serotypes A and C, additional Lys residues within this region may be mutated to either Gln or His, thereby eliminating additional trypsin-susceptible sites (i.e., changing amino acids 438-444 of SEQ ID NO:1 to HTQSLDQG (SEQ ID NO:11) and K₄₄₈>G). As one example, without limitation, the addition of a TEV recognition sequence and the removal of the low specificity protease sites, changes the intermediate region sequence to HTQSLDQGGENLYFQG (SEQ ID NO:12).

Trypsin-susceptible recognition sequences also occur upstream of the heavy chain's receptor-binding domain in serotypes A, E, and F. This region's susceptibility to proteolysis is consistent with its exposure to solvent in the toxin's 3-D structure, as shown by X-ray crystallography analysis. Therefore, in serotypes A, E, and F, the susceptible residues are modified to Asn (i.e., changing amino acid K₈₇₁>N). These modifications enable standardization of activation with either enterokinase or TEV.

Signal peptides and N-terminal affinity tags can also be introduced, as required, to enable secretion and recovery and to eliminate truncated variants. The affinity tags can be separated from the N-terminus and C-terminus of the neurotoxin by cleavage using the same specific proteases used to cleave the heavy and light chain (e.g., enterokinase or TEV cleavage sites).

In one embodiment, the Clostridium botulinum neurotoxin is from a propeptide that has a metalloprotease disabling mutation. The amino acid residues constituting the minimal catalytic domain of the light chain of the propeptide are illustrated in FIG. 1A with a grey background. Specific amino acid residues constituting the active site of the catalytic domain of the metalloprotease are marked by stars in FIG. 1A.

A variety of Clostridial neurotoxin propeptides with light chain regions containing non-native motifs (e.g., SNARE motif peptides) in place of surface alpha-helix domains can be created. These non-native motif bearing propeptides are generated by altering the nucleotide sequences of nucleic acids encoding the propeptides. The sequences of nine non-native motifs that may be substituted for alpha-helix domains are described in U.S. Pat. No. 7,785,606 to Ichtchenko and Band, which is hereby incorporated by reference in its entirety. Neurotoxin derivatives that incorporate sequences to target other cellular receptors are also possible (e.g., EGF or cancer cells) (see U.S. Patent Application Publication No. 2012/0064059 to Foster et al., which is hereby incorporated by reference in its entirety).

In one embodiment, the light and heavy chains of the propeptide are not truncated.

In one embodiment, the propeptide further comprises a signal peptide coupled to the light chain region, where the signal peptide is suitable to permit secretion of the propeptide from a eukaryotic cell to a medium. Suitable signal peptides are described in U.S. Pat. No. 7,785,606 to Ichtchenko and Band, which is hereby incorporated by reference in its entirety. A suitable signal peptide is a gp64 signal peptide. Another suitable signal peptide is MMKFLVNVALVFMVVYISYIYAAG . . . (SEQ ID NO:13). Other signal peptides may also be used. Signal peptides are chosen based on suitability to the expression system being used.

The propeptide may also have an affinity tag located between the signal peptide and the light chain region and/or at the C-terminus of the propeptide after the heavy chain region. A signal peptide with suitable affinity tag such as the hexahistidine affinity tag is MPMLSAIVLYVLLAAAAHSAFAAMVHHHHHHSAS . . . (SEQ ID NO:14). An example of a suitable signal peptide with a longer histidine affinity tag, a TEV cleavage site, and a linker sequence (ATRGAGAG, SEQ ID NO:15) is MKFLVNVALVFMVVYISYIYAAGHTIFITIHHHEITITIHDVENLYFQGATRGAGAG (SEQ ID NO:16). A linker sequence can make the affinity tag accessible. Another example of a suitable affinity tag is a Strep tag II: WSHPQFEK (SEQ ID NO:17). In some embodiments, multiple Strep tag II sequences are used connected by linker sequences (GAG). Any suitable affinity tag may be used. In some embodiments, no affinity tag is used. In some embodiments, the affinity tag and signal peptide are cleaved away from the Clostridial neurotoxin derivative by a high affinity protease site. In some embodiments, the signal peptide is placed between the high affinity protease site and the Clostridial neurotoxin light chain derivative. In some embodiments, affinity tags in multiple locations are cleaved away from the Clostridial neurotoxin derivative molecule by high affinity protease sites. In some embodiments no affinity tag or high affinity protease site is used. In some embodiments a linker is used. In some embodiments, no linker is used. Any suitable linker sequence to connect various elements may be used such as those described in the present application.

Structural variations of suitable Clostridial neurotoxin propeptides that possess a cargo attachment peptide sequence are described in U.S. Patent Application Publication No. 2011/0206616 to Ichtchenko and Band, which is hereby incorporated by reference in its entirety. Propeptides that encode derivatives of a Clostridial neurotoxin suitable for use in the method of the present application may have many of the structural features of the propeptides described in U.S. Patent Application Publication No. 2011/0206616 to Ichtchenko and Band, which is hereby incorporated by reference in its entirety, other than the cargo attachment peptide sequence at the N-terminus. As described in U.S. Patent Application Publication No. 2011/0206616 to Ichtchenko and Band, which is hereby incorporated by reference in its entirety, a single protease cleavage step can be used for activation by simultaneous cleavage between the light chain and heavy chain and removal of affinity tags.

Isolated nucleic acid molecules that encode Clostridium botulinum neurotoxin suitable for use in treatment methods are described in U.S. Pat. No. 7,785,606 to Ichtchenko and Band, and U.S. Pat. No. 9,315,459 to Vasquez-Cintron, Ichtchenko, and Band, which are each hereby incorporated by reference in their entirety. In one embodiment, the nucleic acid molecule has a metalloprotease disabling mutation, as described supra. A nucleotide sequence encoding a BoNT A molecule with a signal peptide, affinity tag, highly specific protease sites, a metalloprotease disabling mutation (Y₃₆₆>F), and C-terminal affinity tags is shown in SEQ ID NO:18, as follows:

GGATCCCGGT CCGCCAGAAC TCTGGAACTC CTAAAAAACC
GCCACCATGA AATTCTTAGT CAACGTTGCC CTTGTTTTTA
TGGTCGTATA CATTTCTTAC ATCTATGCGG CCGGACACCA
TCATCACCAC CACCATCATC ATCACCACGA CGTCGAAAAC
CTGTACTTCC AAGGTGCCAC GCGTGGAGCG GGAGCAGGTC
CATTCGTCAA CAAGCAATTC AACTACAAAG ATCCTGTTAA
CGGTGTGGAC ATCGCCTACA TCAAGATTCC GAACGCAGGC
CAGATGCAAC CTGTGAAGGC TTTCAAAATC CACAACAAGA
TCTGGGTCAT TCCCGAGAGA GACACATTCA CGAACCCAGA
GGAAGGTGAT CTGAACCCTC CCCCAGAAGC CAAGCAGGTG
CCGGTCTCTT ACTACGATTC AACCTACCTC AGTACTGACA
ACGAGAAGGA TAACTACCTG AAGGGCGTTA CTAAACTCTT
CGAGCGCATC TACTCGACAG ACTTGGGCCG TATGCTGCTC
ACGTCCATCG TCAGGGGTAT TCCTTTCTGG GGTGGCTCAA
CCATCGACAC TGAGCTGAAG GTCATTGATA CAAACTGCAT
CAACGTTATT CAACCCGACG GCTCCTACCG CAGCGAGGAA
TTGAACCTGG TGATCATTGG ACCAAGCGCC GACATCATTC
AGTTCGAGTG TAAGTCTTTC GGCCATGAAG TCCTCAACTT
GACCAGAAAC GGCTACGGCT CCACTCAATA CATCCGCTTC
AGCCCCGACT TCACATTCGG ATTCGAGGAA TCACTGGAGG
TCGATACGAA CCCGTTGCTG GGTGCTGGCA AGTTCGCCAC
CGACCCTGCA GTTACTCTGG CACACGAGCT CATCCACGCG
GGACATCGTC TGTACGGTAT CGCTATTAAC CCAAACAGGG
TCTTCAAGGT TAACACCAAC GCCTACTACG AGATGAGTGG
TCTGGAAGTG TCGTTCGAGG AACTCCGTAC GTTCGGAGGT
CACGACGCAA AGTTCATCGA TAGTTTGCAG GAGAACGAAT
TCCGCCTGTA CTACTACAAC AAGTTCAAAG ACATCGCGTC
TACACTCAAC AAGGCTAAAA GCATTGTTGG AACCACTGCT
AGTTTGCAAT ACATGAAGAA CGTGTTCAAG GAGAAATACC
TCTTGTCGGA AGACACCTCC GGTAAATTCA GCGTGGACAA
GCTGAAATTC GATAAGTTGT ACAAAATGCT GACAGAAATC
TACACGGAAG ACAACTTCGT TAAGTTCTTC AAAGTGTTGA
ACCGTAAGAC CTTCCTGAAC TTCGATAAGG CTGTCTTCAA
AATCAACATT GTGCCTAAAG TCAACTACAC CATCTACGAC
GGTTTCAACC TCCGCAACAC TAACTTGGCT GCCAACTTCA
ACGGCCAGAA CACTGAGATC AACAACATGA ACTTCACAAA
GCTCAAAAAC TTCACCGGTT TGTTCGAGTT CTACAAGCTG
CTCTGCGTGC GTGGTATCAT TACATCTCAC ACGCAATCTC
TAGACCAGGG TGGCGAGAAC CTGTACTTCC AGGGTGCTCT
GAACGATCTG TGTATCAAGG TGAATAACTG GGATCTGTTC
TTTAGCCCAA GCGAGGATAA CTTCACGAAC GATCTCAACA
AAGGTGAAGA GATCACGTCT GATACCAATA TCGAAGCGGC
TGAAGAGAAT ATCTCCTTGG ATCTCATCCA GCAATATTAC
CTGACCTTTA ACTTCGATAA CGAGCCCGAA AACATCTCCA
TCGAGAACCT CAGCTCAGAC ATCATTGGTC AGTTGGAGCT
GATGCCAAAC ATTGAACGCT TCCCCAACGG CAAGAAATAC
GAACTCGACA AGTATACGAT GTTTCATTAC TTAAGAGCGC
AGGAGTTTGA ACACGGCAAG AGCCGCATTG CTCTCACTAA
CTCCGTGAAT GAAGCCCTGC TCAATCCGTC AAGGGTGTAC
ACATTCTTTA GCTCCGACTA TGTCAAGAAA GTGAACAAAG
CCACCGAAGC GGCAATGTTC CTGGGATGGG TTGAACAACT
GGTCTACGAC TTCACCGACG AGACCTCTGA GGTGAGCACA
ACGGACAAGA TTGCTGACAT CACTATCATT ATCCCGTATA
TTGGACCTGC CTTGAATATT GGCAACATGC TCTACAAAGA
CGATTTCGTT GGTGCCCTGA TCTTCAGCGG TGCCGTGATC
CTGTTGGAGT TCATTCCTGA AATCGCCATC CCTGTGCTGG
GCACGTTCGC TCTGGTCTCA TACATTGCGA ATAAGGTCTT
GACCGTGCAG ACAATCGATA ATGCCCTCTC CAAACGTAAC
GAAAAATGGG ACGAGGTCTA CAAATACATC GTGACCAACT
GGCTGGCAAA GGTTAACACC CAAATTGATC TGATCCGTAA
GAAAATGAAG GAGGCTTTGG AGAACCAGGC TGAAGCTACT
AAAGCCATTA TCAACTACCA GTATAATCAG TATACAGAAG
AGGAAAAGAA TAACATCAAT TTCAACATCG ATGACTTGTC
CTCAAAGCTG AACGAGTCCA TCAACAAAGC TATGATCAAC
ATCAACAAAT TCCTGAATCA GTGCTCCGTG TCTTACCTGA
TGAACTCTAT GATCCCATAC GGTGTGAAGC GCCTGGAGGA
CTTCGATGCC AGCCTGAAAG ACGCACTGCT CAAATACATT
TACGATAATC GCGGCACTTT GATTGGCCAA GTTGACCGTC
TGAAGGACAA GGTTAACAAT ACCTTGTCAA CCGATATCCC
CTTCCAACTC TCTAAGTACG TCGATAACCA GCGCTTGCTG
AGCACCTTCA CAGAATACAT CAACAACATC ATCAACACCT
CCATCCTGAA CCTCCGTTAC GAGTCTAACC ACCTCATCGA
CTTGAGCAGA TACGCTAGCA AGATCAACAT CGGTTCCAAG
GTGAACTTCG ACCCAATCGA TAAGAACCAG ATCCAACTGT
TCAACCTCGA ATCCTCTAAG ATCGAAGTGA TCCTGAAGAA
CGCTATCGTC TACAACTCCA TGTACGAAAA CTTCTCTACC
AGCTTCTGGA TCAGGATTCC GAAATACTTC AACTCAATCT
CGCTCAACAA CGAGTACACT ATCATCAACT GCATGGAAAA
CAACTCGGGA TGGAAGGTGT CCCTCAACTA CGGCGAGATC
ATCTGGACTT TGCAGGACAC ACAAGAAATC AAGCAGAGGG
TCGTGTTCAA GTACAGCCAA ATGATCAACA TCAGCGATTA
CATCAACCGT TGGATCTTCG TCACAATCAC CAACAACCGC
CTGAACAACT CCAAGATTTA CATCAACGGT AGACTGATCG
ACCAGAAGCC AATCAGCAAC CTCGGCAACA TCCACGCCTC
AAACAACATC ATGTTCAAGT TGGACGGCTG TAGGGATACA
CACAGATACA TCTGGATCAA ATACTTCAAC CTGTTCGACA
AGGAGCTCAA CGAGAAGGAA ATCAAGGACC TCTACGATAA
CCAGTCCAAC TCTGGTATCT TGAAGGACTT CTGGGGCGAT
TACCTGCAAT ACGACAAGCC CTACTACATG TTGAACCTGT
ACGACCCTAA CAAGTACGTT GATGTGAACA ACGTCGGTAT
CAGGGGCTAC ATGTACCTGA AGGGACCACG TGGTTCTGTT
ATGACCACTA ACATCTACCT CAACAGCTCA TTGTACCGTG
GCACAAAGTT CATCATCAAG AAGTACGCCT CCGGAAACAA
GGACAACATC GTCCGTAACA ACGATCGCGT TTACATCAAC
GTTGTGGTCA AGAACAAGGA GTACAGACTG GCTACCAACG
CTTCGCAGGC TGGAGTTGAG AAGATCCTGT CTGCTCTGGA
AATCCCTGAC GTGGGCAACC TCTCACAGGT TGTGGTCATG
AAGTCGAAGA ACGATCAAGG CATCACTAAC AAGTGCAAGA
TGAACTTGCA GGACAACAAC GGAAACGACA TCGGCTTCAT
CGGATTCCAC CAATTCAACA ACATCGCCAA GTTGGTGGCC
AGCAACTGGT ACAACCGTCA GATCGAGCGT TCGTCCCGCA
CCTTAGGATG CTCGTGGGAG TTCATTCCAG TCGATGACGG
ATGGGGAGAG AGACCTTTGG GCGCAGGAGA GAACCTGTAC
TTCCAGGGTG CAGGATGGTC CCACCCACAA TTCGAGAAGG
GTGCAGGATG GAGTCACCCA CAGTTCGAGA AGGGCGCTGG
ATGGTCCCAC CCACAGTTCG AGAAATAATT AGTTGATGCA
TAGTTAATTA GATAGCTCGA G

A nucleotide sequence encoding a BoNT A molecule with a signal peptide, affinity tag, highly specific protease sites, a metalloprotease disabling mutation (E₂₂₄>Q), and C-terminal affinity tags is shown in SEQ ID NO:19:

GGATCCCGGT CCGCCAGAAC TCTGGAACTC CTAAAAAACC
GCCACCATGA AATTCTTAGT CAACGTTGCC CTTGTTTTTA
TGGTCGTATA CATTTCTTAC ATCTATGCGG CCGGACACCA
TCATCACCAC CACCATCATC ATCACCACGA CGTCGAAAAC
CTGTACTTCC AAGGTGCCAC GCGTGGAGCG GGAGCAGGTC
CATTCGTCAA CAAGCAATTC AACTACAAAG ATCCTGTTAA
CGGTGTGGAC ATCGCCTACA TCAAGATTCC GAACGCAGGC
CAGATGCAAC CTGTGAAGGC TTTCAAAATC CACAACAAGA
TCTGGGTCAT TCCCGAGAGA GACACATTCA CGAACCCAGA
GGAAGGTGAT CTGAACCCTC CCCCAGAAGC CAAGCAGGTG
CCGGTCTCTT ACTACGATTC AACCTACCTC AGTACTGACA
ACGAGAAGGA TAACTACCTG AAGGGCGTTA CTAAACTCTT
CGAGCGCATC TACTCGACAG ACTTGGGCCG TATGCTGCTC
ACGTCCATCG TCAGGGGTAT TCCTTTCTGG GGTGGCTCAA
CCATCGACAC TGAGCTGAAG GTCATTGATA CAAACTGCAT
CAACGTTATT CAACCCGACG GCTCCTACCG CAGCGAGGAA
TTGAACCTGG TGATCATTGG ACCAAGCGCC GACATCATTC
AGTTCGAGTG TAAGTCTTTC GGCCATGAAG TCCTCAACTT
GACCAGAAAC GGCTACGGCT CCACTCAATA CATCCGCTTC
AGCCCCGACT TCACATTCGG ATTCGAGGAA TCACTGGAGG
TCGATACGAA CCCGTTGCTG GGTGCTGGCA AGTTCGCCAC
CGACCCTGCA GTTACTCTGG CACACCAGCT CATCCACGCG
GGACATCGTC TGTACGGTAT CGCTATTAAC CCAAACAGGG
TCTTCAAGGT TAACACCAAC GCCTACTACG AGATGAGTGG
TCTGGAAGTG TCGTTCGAGG AACTCCGTAC GTTCGGAGGT
CACGACGCAA AGTTCATCGA TAGTTTGCAG GAGAACGAAT
TCCGCCTGTA CTACTACAAC AAGTTCAAAG ACATCGCGTC
TACACTCAAC AAGGCTAAAA GCATTGTTGG AACCACTGCT
AGTTTGCAAT ACATGAAGAA CGTGTTCAAG GAGAAATACC
TCTTGTCGGA AGACACCTCC GGTAAATTCA GCGTGGACAA
GCTGAAATTC GATAAGTTGT ACAAAATGCT GACAGAAATC
TACACGGAAG ACAACTTCGT TAAGTTCTTC AAAGTGTTGA
ACCGTAAGAC CTACCTGAAC TTCGATAAGG CTGTCTTCAA
AATCAACATT GTGCCTAAAG TCAACTACAC CATCTACGAC
GGTTTCAACC TCCGCAACAC TAACTTGGCT GCCAACTTCA
ACGGCCAGAA CACTGAGATC AACAACATGA ACTTCACAAA
GCTCAAAAAC TTCACCGGTT TGTTCGAGTT CTACAAGCTG
CTCTGCGTGC GTGGTATCAT TACATCTCAC ACGCAATCTC
TAGACCAGGG TGGCGAGAAC CTGTACTTCC AGGGTGCTCT
GAACGATCTG TGTATCAAGG TGAATAACTG GGATCTGTTC
TTTAGCCCAA GCGAGGATAA CTTCACGAAC GATCTCAACA
AAGGTGAAGA GATCACGTCT GATACCAATA TCGAAGCGGC
TGAAGAGAAT ATCTCCTTGG ATCTCATCCA GCAATATTAC
CTGACCTTTA ACTTCGATAA CGAGCCCGAA AACATCTCCA
TCGAGAACCT CAGCTCAGAC ATCATTGGTC AGTTGGAGCT
GATGCCAAAC ATTGAACGCT TCCCCAACGG CAAGAAATAC
GAACTCGACA AGTATACGAT GTTTCATTAC TTAAGAGCGC
AGGAGTTTGA ACACGGCAAG AGCCGCATTG CTCTCACTAA
CTCCGTGAAT GAAGCCCTGC TCAATCCGTC AAGGGTGTAC
ACATTCTTTA GCTCCGACTA TGTCAAGAAA GTGAACAAAG
CCACCGAAGC GGCAATGTTC CTGGGATGGG TTGAACAACT
GGTCTACGAC TTCACCGACG AGACCTCTGA GGTGAGCACA
ACGGACAAGA TTGCTGACAT CACTATCATT ATCCCGTATA
TTGGACCTGC CTTGAATATT GGCAACATGC TCTACAAAGA
CGATTTCGTT GGTGCCCTGA TCTTCAGCGG TGCCGTGATC
CTGTTGGAGT TCATTCCTGA AATCGCCATC CCTGTGCTGG
GCACGTTCGC TCTGGTCTCA TACATTGCGA ATAAGGTCTT
GACCGTGCAG ACAATCGATA ATGCCCTCTC CAAACGTAAC
GAAAAATGGG ACGAGGTCTA CAAATACATC GTGACCAACT
GGCTGGCAAA GGTTAACACC CAAATTGATC TGATCCGTAA
GAAAATGAAG GAGGCTTTGG AGAACCAGGC TGAAGCTACT
AAAGCCATTA TCAACTACCA GTATAATCAG TATACAGAAG
AGGAAAAGAA TAACATCAAT TTCAACATCG ATGACTTGTC
CTCAAAGCTG AACGAGTCCA TCAACAAAGC TATGATCAAC
ATCAACAAAT TCCTGAATCA GTGCTCCGTG TCTTACCTGA
TGAACTCTAT GATCCCATAC GGTGTGAAGC GCCTGGAGGA
CTTCGATGCC AGCCTGAAAG ACGCACTGCT CAAATACATT
TACGATAATC GCGGCACTTT GATTGGCCAA GTTGACCGTC
TGAAGGACAA GGTTAACAAT ACCTTGTCAA CCGATATCCC
CTTCCAACTC TCTAAGTACG TCGATAACCA GCGCTTGCTG
AGCACCTTCA CAGAATACAT CAACAACATC ATCAACACCT
CCATCCTGAA CCTCCGTTAC GAGTCTAACC ACCTCATCGA
CTTGAGCAGA TACGCTAGCA AGATCAACAT CGGTTCCAAG
GTGAACTTCG ACCCAATCGA TAAGAACCAG ATCCAACTGT
TCAACCTCGA ATCCTCTAAG ATCGAAGTGA TCCTGAAGAA
CGCTATCGTC TACAACTCCA TGTACGAAAA CTTCTCTACC
AGCTTCTGGA TCAGGATTCC GAAATACTTC AACTCAATCT
CGCTCAACAA CGAGTACACT ATCATCAACT GCATGGAAAA
CAACTCGGGA TGGAAGGTGT CCCTCAACTA CGGCGAGATC
ATCTGGACTT TGCAGGACAC ACAAGAAATC AAGCAGAGGG
TCGTGTTCAA GTACAGCCAA ATGATCAACA TCAGCGATTA
CATCAACCGT TGGATCTTCG TCACAATCAC CAACAACCGC
CTGAACAACT CCAAGATTTA CATCAACGGT AGACTGATCG
ACCAGAAGCC AATCAGCAAC CTCGGCAACA TCCACGCCTC
AAACAACATC ATGTTCAAGT TGGACGGCTG TAGGGATACA
CACAGATACA TCTGGATCAA ATACTTCAAC CTGTTCGACA
AGGAGCTCAA CGAGAAGGAA ATCAAGGACC TCTACGATAA
CCAGTCCAAC TCTGGTATCT TGAAGGACTT CTGGGGCGAT
TACCTGCAAT ACGACAAGCC CTACTACATG TTGAACCTGT
ACGACCCTAA CAAGTACGTT GATGTGAACA ACGTCGGTAT
CAGGGGCTAC ATGTACCTGA AGGGACCACG TGGTTCTGTT
ATGACCACTA ACATCTACCT CAACAGCTCA TTGTACCGTG
GCACAAAGTT CATCATCAAG AAGTACGCCT CCGGAAACAA
GGACAACATC GTCCGTAACA ACGATCGCGT TTACATCAAC
GTTGTGGTCA AGAACAAGGA GTACAGACTG GCTACCAACG
CTTCGCAGGC TGGAGTTGAG AAGATCCTGT CTGCTCTGGA
AATCCCTGAC GTGGGCAACC TCTCACAGGT TGTGGTCATG
AAGTCGAAGA ACGATCAAGG CATCACTAAC AAGTGCAAGA
TGAACTTGCA GGACAACAAC GGAAACGACA TCGGCTTCAT
CGGATTCCAC CAATTCAACA ACATCGCCAA GTTGGTGGCC
AGCAACTGGT ACAACCGTCA GATCGAGCGT TCGTCCCGCA
CCTTAGGATG CTCGTGGGAG TTCATTCCAG TCGATGACGG
ATGGGGAGAG AGACCTTTGG GCGCAGGAGA GAACCTGTAC
TTCCAGGGTG CAGGATGGTC CCACCCACAA TTCGAGAAGG
GTGCAGGATG GAGTCACCCA CAGTTCGAGA AGGGCGCTGG
ATGGTCCCAC CCACAGTTCG AGAAATAATT AGTTGATGCA
TAGTTAATTA GATAGCTCGA G

Expression systems having a nucleic acid molecule encoding an isolated, Clostridium botulinum neurotoxin in a heterologous vector, and host cells having a heterologous nucleic acid molecule encoding Clostridium botulinum neurotoxins are described in U.S. Patent No. 7,785,606 to Ichtchenko and Band, and U.S. Pat. No. 9,315,459 to Vasquez-Cintron, Ichtchenko, and Band, which are each hereby incorporated by reference in their entirety.

Expressing a recombinant, physiologically active, Clostridium botulinum neurotoxins is carried out by providing a nucleic acid construct having a nucleic acid molecule encoding a propeptide as described herein. The nucleic acid construct has a heterologous promoter operably linked to the nucleic acid molecule and a 3′ regulatory region operably linked to the nucleic acid molecule. The nucleic acid construct is then introduced into a host cell under conditions effective to express the Clostridium botulinum neurotoxin.

Expression of a Clostridium botulinum neurotoxin can be carried out by introducing a nucleic acid molecule encoding a propeptide (e.g., SEQ ID NO:18 or 19) into an expression system of choice using conventional recombinant technology. Generally, this involves inserting the nucleic acid molecule into an expression system to which the molecule is heterologous (i.e., not normally present). The introduction of a particular foreign or native gene into a mammalian host is facilitated by first introducing the gene sequence into a suitable nucleic acid vector. “Vector” is used herein to mean any genetic element, such as a plasmid, phage, transposon, cosmid, chromosome, virus, virion, etc., which is capable of replication when associated with the proper control elements and which is capable of transferring gene sequences between cells. Thus, the term includes cloning and expression vectors, as well as viral vectors. The heterologous nucleic acid molecule is inserted into the expression system or vector in proper sense (5′→3′) orientation and correct reading frame. The vector contains the necessary elements for the transcription and translation of the inserted Clostridial neurotoxin propeptide-coding sequences.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in culture.

Recombinant genes may also be introduced into viruses, including vaccinia virus, adenovirus, and retroviruses, including lentivirus. Recombinant viruses can be generated by transfection of plasmids into cells infected with virus.

Suitable vectors include, but are not limited to, the following viral vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK +/− or KS +/− (see “Stratagene Cloning Systems” Catalog (1993) from Stratagene, La Jolla, Calif., which is hereby incorporated by reference in its entirety), pQE, pIH821, pGEX, pFastBac series (Invitrogen), pET series (see F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Cold Springs Laboratory, Cold Springs Harbor, New York (1989), which is hereby incorporated by reference in its entirety.

A variety of host-vector systems may be utilized to express the propeptide-encoding sequence in a cell. Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (“mRNA”) translation).

Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzymology 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the PH promoter, T7 phage promoter, lac promoter, trp promoter, recA promoter, ribosomal RNA promoter, the P_{R} and P_{L} promoters of coliphage lambda and others, including but not limited, to lacUV5, ompF, bla, 1pp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno (“SD”) sequence about 7-9 bases 5′ to the initiation codon (ATG) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Depending on the vector system and host utilized, any number of suitable transcription and/or translation elements, including constitutive, inducible, and repressible promoters, as well as minimal 5′ promoter elements may be used.

The nucleic acid, a promoter molecule of choice, a suitable 3′ regulatory region, and if desired, a reporter gene, are incorporated into a vector-expression system of choice to prepare a nucleic acid construct using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

The nucleic acid molecule encoding a derivative of a Clostridial neurotoxin is inserted into a vector in the sense (i.e., 5′→3′) direction, such that the open reading frame is properly oriented for the expression of the encoded propeptide under the control of a promoter of choice. Single or multiple nucleic acids may be ligated into an appropriate vector in this way, under the control of a suitable promoter, to prepare a nucleic acid construct. For example, the nucleotide sequence of SEQ ID NOs: 18 or 19 can be cloned into insect vector pFASTBacI using restriction enzymes and standard methods. See, e.g., standard procedures in the art as described by Sambrook et al., Molecular Cloning: A Laboratory Manual, Second Edition, Cold Spring Harbor, N.Y., Cold Spring Harbor Press (1989), and Ausubel et al., Current Protocols in Molecular Biology, New York, N.Y., John Wiley & Sons (1989), which are hereby incorporated by reference in their entirety.

Once the isolated nucleic acid molecule encoding the propeptide has been inserted into an expression vector, it is ready to be incorporated into a host cell. Recombinant molecules can be introduced into cells via transformation, particularly transduction, conjugation, lipofection, protoplast fusion, mobilization, particle bombardment, or electroporation. The DNA sequences are incorporated into the host cell using standard cloning procedures known in the art, as described by Sambrook et al., Molecular Cloning: A Laboratory Manual, Second Edition, Cold Springs Laboratory, Cold Springs Harbor, New York (1989), which is hereby incorporated by reference in its entirety. Suitable hosts include, but are not limited to, bacteria, virus, yeast, fungi, mammalian cells, insect cells, plant cells, and the like. Preferable host cells of the present application include, but are not limited to, Escherichia coli, insect cells, and Pichia pastoris cells.

Typically, an antibiotic or other compound useful for selective growth of the transformed cells only is added as a supplement to the media. The compound to be used will be dictated by the selectable marker element present in the plasmid with which the host cell was transformed. Suitable genes are those which confer resistance to gentamycin, G418, hygromycin, puromycin, streptomycin, spectinomycin, tetracycline, chloramphenicol, and the like. Similarly, “reporter genes” which encode enzymes providing for production of an identifiable compound, or other markers which indicate relevant information regarding the outcome of gene delivery, are suitable. For example, various luminescent or phosphorescent reporter genes are also appropriate, such that the presence of the heterologous gene may be ascertained visually.

In some embodiments, the expressed neurotoxin derivative is secreted via the signal peptide. In some embodiments, the neurotoxin derivative is purified using one or more affinity tags. In some embodiments, the expressed neurotoxin derivative is contacted with a highly specific protease under conditions effective to effect cleavage at the intermediate region. In some embodiments, the intermediate region of the propeptide is not cleaved by proteases endogenous to the expression system or the host cell. In some embodiments, the expressed neurotoxin derivative is contacted with a highly specific protease under conditions effective to effect cleavage at the intermediate region, prior to the light chain and after the heavy chain of the Costridial neurotoxin derivative. Exemplary neurotoxin derivatives after TEV cleavage are as follows:

A Clostridial neurotoxin derivative propeptide amino acid sequence derived from SEQ ID NO:18 having a metalloprotease in the light chain with a mutation corresponding to Y₃₆₆>F in BoNT A is SEQ ID NO:20, as follows:

MKFLVNVALV FMVVYISYIY AAGHHHHHHH HHHHDVENLY
FQGATRGAGA GPFVNKQFNY KDPVNGVDIA YIKIPNAGQM
QPVKAFKIHN KIWVIPERDT FTNPEEGDLN PPPEAKQVPV
SYYDSTYLST DNEKDNYLKG VTKLFERIYS TDLGRMLLTS
IVRGIPFWGG STIDTELKVI DTNCINVIQP DGSYRSEELN
LVIIGPSADI IQFECKSFGH EVLNLTRNGY GSTQYIRFSP
DFTFGFEESL EVDTNPLLGA GKFATDPAVT LAHELIHAGH
RLYGIAINPN RVFKVNTNAY YEMSGLEVSF EELRTFGGHD
AKFIDSLQEN EFRLYYYNKF KDIASTLNKA KSIVGTTASL
QYMKNVFKEK YLLSEDTSGK FSVDKLKFDK LYKMLTEIYT
EDNFVKFFKV LNRKTFLNFD KAVFKINIVP KVNYTIYDGF
NLRNTNLAAN FNGQNTEINN MNFTKLKNFT GLFEFYKLLC
VRGIITSHTQ SLDQGGENLY FQGALNDLCI KVNNWDLFFS
PSEDNFTNDL NKGEEITSDT NIEAAEENIS LDLIQQYYLT
FNFDNEPENI SIENLSSDII GQLELMPNIE RFPNGKKYEL
DKYTMFHYLR AQEFEHGKSR IALTNSVNEA LLNPSRVYTF
FSSDYVKKVN KATEAAMFLG WVEQLVYDFT DETSEVSTTD
KIADITIIIP YIGPALNIGN MLYKDDFVGA LIFSGAVILL
EFIPEIAIPV LGTFALVSYI ANKVLTVQTI DNALSKRNEK
WDEVYKYIVT NWLAKVNTQI DLIRKKMKEA LENQAEATKA
IINYQYNQYT EEEKNNINFN IDDLSSKLNE SINKAMININ
KFLNQCSVSY LMNSMIPYGV KRLEDFDASL KDALLKYIYD
NRGTLIGQVD RLKDKVNNTL STDIPFQLSK YVDNQRLLST
FTEYINNIIN TSILNLRYES NHLIDLSRYA SKINIGSKVN
FDPIDKNQIQ LFNLESSKIE VILKNAIVYN SMYENFSTSF
WIRIPKYFNS ISLNNEYTII NCMENNSGWK VSLNYGEIIW
TLQDTQEIKQ RVVFKYSQMI NISDYINRWI FVTITNNRLN
NSKIYINGRL IDQKPISNLG NIHASNNIMF KLDGCRDTHR
YIWIKYFNLF DKELNEKEIK DLYDNQSNSG ILKDFWGDYL
QYDKPYYMLN LYDPNKYVDV NNVGIRGYMY LKGPRGSVMT
TNIYLNSSLY RGTKFIIKKY ASGNKDNIVR NNDRVYINVV
VKNKEYRLAT NASQAGVEKI LSALEIPDVG NLSQVVVMKS
KNDQGITNKC KMNLQDNNGN DIGFIGFHQF NNIAKLVASN
WYNRQIERSS RTLGCSWEFI PVDDGWGERP LGAGENLYFQ
GAGWSHPQFE KGAGWSHPQF EKGAGWSHPQ FEK

After cleavage of SEQ ID NO:20 with a highly specific protease such as TEV, the active Clostridial neurotoxin derivative may, according to one embodiment, have a light chain sequence as shown in SEQ ID NO:21, as follows:

GATRGAGAGP FVNKQFNYKD PVNGVDIAYI KIPNAGQMQP
VKAFKIHNKI WVIPERDTFT NPEEGDLNPP PEAKQVPVSY
YDSTYLSTDN EKDNYLKGVT KLFERIYSTD LGRMLLTSIV
RGIPFWGGST IDTELKVIDT NCINVIQPDG SYRSEELNLV
IIGPSADIIQ FECKSFGHEV LNLTRNGYGS TQYIRFSPDF
TFGFEESLEV DTNPLLGAGK FATDPAVTLA HELIHAGHRL
YGIAINPNRV FKVNTNAYYE MSGLEVSFEE LRTFGGHDAK
FIDSLQENEF RLYYYNKFKD IASTLNKAKS IVGTTASLQY
MKNVFKEKYL LSEDTSGKFS VDKLKFDKLY KMLTEIYTED
NFVKFFKVLN RKTFLNFDKA VFKINIVPKV NYTIYDGFNL
RNTNLAANFN GQNTEINNMN FTKLKNFTGL FEFYKLLCVR
GIITSHTQSL DQGGENLYFQ

After cleavage of SEQ ID NO:20 with a highly specific protease such as TEV, the active Clostridial neurotoxin derivative may, according to one embodiment, have a heavy chain sequence as shown in SEQ ID NO:22, as follows:

GALNDLCIKV NNWDLFFSPS EDNFTNDLNK GEEITSDTNI
EAAEENISLD LIQQYYLTFN FDNEPENISI ENLSSDIIGQ
LELMPNIERF PNGKKYELDK YTMFHYLRAQ EFEHGKSRIA
LTNSVNEALL NPSRVYTFFS SDYVKKVNKA TEAAMFLGWV
EQLVYDFTDE TSEVSTTDKI ADITIIIPYI GPALNIGNML
YKDDFVGALI FSGAVILLEF IPEIAIPVLG TFALVSYIAN
KVLTVQTIDN ALSKRNEKWD EVYKYIVTNW LAKVNTQIDL
IRKKMKEALE NQAEATKAII NYQYNQYTEE EKNNINFNID
DLSSKLNESI NKAMININKF LNQCSVSYLM NSMIPYGVKR
LEDFDASLKD ALLKYIYDNR GTLIGQVDRL KDKVNNTLST
DIPFQLSKYV DNQRLLSTFT EYINNIINTS ILNLRYESNH
LIDLSRYASK INIGSKVNFD PIDKNQIQLF NLESSKIEVI
LKNAIVYNSM YENFSTSFWI RIPKYFNSIS LNNEYTIINC
MENNSGWKVS LNYGEIIWTL QDTQEIKQRV VFKYSQMINI
SDYINRWIFV TITNNRLNNS KIYINGRLID QKPISNLGNI
HASNNIMFKL DGCRDTHRYI WIKYFNLFDK ELNEKEIKDL
YDNQSNSGIL KDFWGDYLQY DKPYYMLNLY DPNKYVDVNN
VGIRGYMYLK GPRGSVMTTN IYLNSSLYRG TKFIIKKYAS
GNKDNIVRNN DRVYINVVVK NKEYRLATNA SQAGVEKILS
ALEIPDVGNL SQVVVMKSKN DQGITNKCKM NLQDNNGNDI
GFIGFHQFNN IAKLVASNWY NRQIERSSRT LGCSWEFIPV
DDGWGERPLG AGENLYFQ

The light chain and heavy chain derivatives of SEQ ID NO:21 and SEQ ID NO:22 are linked through disulfide bonds at C₄₃₈ of SEQ ID NO:21 and C₇ of SEQ ID NO:22 to form an active Clostridial neurotoxin derivative. In some embodiments, the light chain derivative is at least 60%, 70%, 80%, 85%, 90%, 95%, 96%, 97%, 98%, or 99% identical to a SEQ ID NO:21. In some embodiments, the heavy chain derivative is at least 60%, 70%, 80%, 85%, 90%, 95%, 96%, 97%, 98%, or 99% identical to SEQ ID NO:22.

Since different types of signal peptides, tags, protease sites, and the orientation of these elements may vary in different embodiments, an exemplary sequence for a light chain derivative, according to the methods disclosed in the present application, is as follows in SEQ ID NO:23:

PFVNKQFNYK DPVNGVDIAY IKIPNAGQMQ PVKAFKIHNK
IWVIPERDTF TNPEEGDLNP PPEAKQVPVS YYDSTYLSTD
NEKDNYLKGV TKLFERIYST DLGRMLLTSI VRGIPFWGGS
TIDTELKVID TNCINVIQPD GSYRSEELNL VIIGPSADII
QFECKSFGHE VLNLTRNGYG STQYIRFSPD FTFGFEESLE
VDTNPLLGAG KFATDPAVTL AHELIHAGHR LYGIAINPNR
VFKVNTNAYY EMSGLEVSFE ELRTFGGHDA KFIDSLQENE
FRLYYYNKFK DIASTLNKAK SIVGTTASLQ YMKNVFKEKY
LLSEDTSGKF SVDKLKFDKL YKMLTEIYTE DNFVKFFKVL
NRKTFLNFDK AVFKINIVPK VNYTIYDGFN LRNTNLAANF
NGQNTEINNM NFTKLKNFTG LFEFYKLLCV RGIITSHTQS
LDQGG

Since different types of signal peptides, tags, protease sites, and the orientation of these elements may vary in different embodiments, an exemplary minimal sequence for a heavy chain derivative, according to the methods disclosed in the present application, is as follows in

SEQ ID NO:24:

ALNDLCIKVN NWDLFFSPSE DNFTNDLNKG EEITSDTNIE
AAEENISLDL IQQYYLTFNF DNEPENISIE NLSSDIIGQL
ELMPNIERFP NGKKYELDKY TMFHYLRAQE FEHGKSRIAL
TNSVNEALLN PSRVYTFFSS DYVKKVNKAT EAAMFLGWVE
QLVYDFTDET SEVSTTDKIA DITIIIPYIG PALNIGNMLY
KDDFVGALIF SGAVILLEFI PEIAIPVLGT FALVSYIANK
VLTVQTIDNA LSKRNEKWDE VYKYIVTNWL AKVNTQIDLI
RKKMKEALEN QAEATKAIIN YQYNQYTEEE KNNINFNIDD
LSSKLNESIN KAMININKFL NQCSVSYLMN SMIPYGVKRL
EDFDASLKDA LLKYIYDNRG TLIGQVDRLK DKVNNTLSTD
IPFQLSKYVD NQRLLSTFTE YINNIINTSI LNLRYESNHL
IDLSRYASKI NIGSKVNFDP IDKNQIQLFN LESSKIEVIL
KNAIVYNSMY ENFSTSFWIR IPKYFNSISL NNEYTIINCM
ENNSGWKVSL NYGEIIWTLQ DTQEIKQRVV FKYSQMINIS
DYINRWIFVT ITNNRLNNSK IYINGRLIDQ KPISNLGNIH
ASNNIMFKLD GCRDTHRYIW IKYFNLFDKE LNEKEIKDLY
DNQSNSGILK DFWGDYLQYD KPYYMLNLYD PNKYVDVNNV
GIRGYMYLKG PRGSVMTTNI YLNSSLYRGT KFIIKKYASG
NKDNIVRNND RVYINVVVKN KEYRLATNAS QAGVEKILSA
LEIPDVGNLS QVVVMKSKND QGITNKCKMN LQDNNGNDIG
FIGFHQFNNI AKLVASNWYN RQIERSSRTL GCSWEFIPVD
DGWGERPL

The light chain and heavy chain derivatives of SEQ ID NO:23 and SEQ ID NO:24 are linked through disulfide bonds at C₄₂₉ of SEQ ID NO:23 and C₆ of SEQ ID NO:24 to form an active Clostridial neurotoxin derivative. In some embodiments, the light chain derivative is at least 60%, 70%, 80%, 85%, 90%, 95%, 96%, 97%, 98%, or 99% identical to a SEQ ID NO:23. In some embodiments, the heavy chain derivative is at least 60%, 70%, 80%, 85%, 90%, 95%, 96%, 97%, 98%, or 99% identical to SEQ ID NO:24.

A Clostridial neurotoxin derivative propeptide amino acid sequence derived from SEQ ID NO: 19 having a metalloprotease in the light chain with a mutation corresponding to an E₂₂₄>Q in BoNT A is SEQ ID NO:25, as follows:

MKFLVNVALV FMVVYISYIY AAGHHHHHHH HHHHDVENLY
FQGATRGAGA GPFVNKQFNY KDPVNGVDIA YIKIPNAGQM
QPVKAFKIHN KIWVIPERDT FTNPEEGDLN PPPEAKQVPV
SYYDSTYLST DNEKDNYLKG VTKLFERIYS TDLGRMLLTS
IVRGIPFWGG STIDTELKVI DTNCINVIQP DGSYRSEELN
LVIIGPSADI IQFECKSFGH EVLNLTRNGY GSTQYIRFSP
DFTFGFEESL EVDTNPLLGA GKFATDPAVT LAHQLIHAGH
RLYGIAINPN RVFKVNTNAY YEMSGLEVSF EELRTFGGHD
AKFIDSLQEN EFRLYYYNKF KDIASTLNKA KSIVGTTASL
QYMKNVFKEK YLLSEDTSGK FSVDKLKFDK LYKMLTEIYT
EDNFVKFFKV LNRKTYLNFD KAVFKINIVP KVNYTIYDGF
NLRNTNLAAN FNGQNTEINN MNFTKLKNFT GLFEFYKLLC
VRGIITSHTQ SLDQGGENLY FQGALNDLCI KVNNWDLFFS
PSEDNFTNDL NKGEEITSDT NIEAAEENIS LDLIQQYYLT
FNFDNEPENI SIENLSSDII GQLELMPNIE RFPNGKKYEL
DKYTMFHYLR AQEFEHGKSR IALTNSVNEA LLNPSRVYTF
FSSDYVKKVN KATEAAMFLG WVEQLVYDFT DETSEVSTTD
KIADITIIIP YIGPALNIGN MLYKDDFVGA LIFSGAVILL
EFIPEIAIPV LGTFALVSYI ANKVLTVQTI DNALSKRNEK
WDEVYKYIVT NWLAKVNTQI DLIRKKMKEA LENQAEATKA
IINYQYNQYT EEEKNNINFN IDDLSSKLNE SINKAMININ
KFLNQCSVSY LMNSMIPYGV KRLEDFDASL KDALLKYIYD
NRGTLIGQVD RLKDKVNNTL STDIPFQLSK YVDNQRLLST
FTEYINNIIN TSILNLRYES NHLIDLSRYA SKINIGSKVN
FDPIDKNQIQ LFNLESSKIE VILKNAIVYN SMYENFSTSF
WIRIPKYFNS ISLNNEYTII NCMENNSGWK VSLNYGEIIW
TLQDTQEIKQ RVVFKYSQMI NISDYINRWI FVTITNNRLN
NSKIYINGRL IDQKPISNLG NIHASNNIMF KLDGCRDTHR
YIWIKYFNLF DKELNEKEIK DLYDNQSNSG ILKDFWGDYL
QYDKPYYMLN LYDPNKYVDV NNVGIRGYMY LKGPRGSVMT
TNIYLNSSLY RGTKFIIKKY ASGNKDNIVR NNDRVYINVV
VKNKEYRLAT NASQAGVEKI LSALEIPDVG NLSQVVVMKS
KNDQGITNKC KMNLQDNNGN DIGFIGFHQF NNIAKLVASN
WYNRQIERSS RTLGCSWEFI PVDDGWGERP LGAGENLYFQ
GAGWSHPQFE KGAGWSHPQF EKGAGWSHPQ FEK

After cleavage of SEQ ID NO:25 with a highly specific protease such as TEV, the active Clostridial neurotoxin derivative may, according to one embodiment, have a light chain sequence as shown in SEQ ID NO:26, as follows:

GATRGAGAGP FVNKQFNYKD PVNGVDIAYI KIPNAGQMQP
VKAFKIHNKI WVIPERDTFT NPEEGDLNPP PEAKQVPVSY
YDSTYLSTDN EKDNYLKGVT KLFERIYSTD LGRMLLTSIV
RGIPFWGGST IDTELKVIDT NCINVIQPDG SYRSEELNLV
IIGPSADIIQ FECKSFGHEV LNLTRNGYGS TQYIRFSPDF
TFGFEESLEV DTNPLLGAGK FATDPAVTLA HQLIHAGHRL
YGIAINPNRV FKVNTNAYYE MSGLEVSFEE LRTFGGHDAK
FIDSLQENEF RLYYYNKFKD IASTLNKAKS IVGTTASLQY
MKNVFKEKYL LSEDTSGKFS VDKLKFDKLY KMLTEIYTED
NFVKFFKVLN RKTYLNFDKA VFKINIVPKV NYTIYDGFNL
RNTNLAANFN GQNTEINNMN FTKLKNFTGL FEFYKLLCVR
GIITSHTQSL DQGGENLYFQ

After cleavage of SEQ ID NO:25 with a highly specific protease such as TEV, the active Clostridial neurotoxin derivative may, according to one embodiment, have a heavy chain sequence as shown in SEQ ID NO:27, as follows:

GALNDLCIKV NNWDLFFSPS EDNFTNDLNK GEEITSDTNI
EAAEENISLD LIQQYYLTFN FDNEPENISI ENLSSDIIGQ
LELMPNIERF PNGKKYELDK YTMFHYLRAQ EFEHGKSRIA
LTNSVNEALL NPSRVYTFFS SDYVKKVNKA TEAAMFLGWV
EQLVYDFTDE TSEVSTTDKI ADITIIIPYI GPALNIGNML
YKDDFVGALI FSGAVILLEF IPEIAIPVLG TFALVSYIAN
KVLTVQTIDN ALSKRNEKWD EVYKYIVTNW LAKVNTQIDL
IRKKMKEALE NQAEATKAII NYQYNQYTEE EKNNINFNID
DLSSKLNESI NKAMININKF LNQCSVSYLM NSMIPYGVKR
LEDFDASLKD ALLKYIYDNR GTLIGQVDRL KDKVNNTLST
DIPFQLSKYV DNQRLLSTFT EYINNIINTS ILNLRYESNH
LIDLSRYASK INIGSKVNFD PIDKNQIQLF NLESSKIEVI
LKNAIVYNSM YENFSTSFWI RIPKYFNSIS LNNEYTIINC
MENNSGWKVS LNYGEIIWTL QDTQEIKQRV VFKYSQMINI
SDYINRWIFV TITNNRLNNS KIYINGRLID QKPISNLGNI
HASNNIMFKL DGCRDTHRYI WIKYFNLFDK ELNEKEIKDL
YDNQSNSGIL KDFWGDYLQY DKPYYMLNLY DPNKYVDVNN
VGIRGYMYLK GPRGSVMTTN IYLNSSLYRG TKFIIKKYAS
GNKDNIVRNN DRVYINVVVK NKEYRLATNA SQAGVEKILS
ALEIPDVGNL SQVVVMKSKN DQGITNKCKM NLQDNNGNDI
GFIGFHQFNN IAKLVASNWY NRQIERSSRT LGCSWEFIPV
DDGWGERPLG AGENLYFQ

The light chain and heavy chain derivatives of SEQ ID NO:26 and SEQ ID NO:27 are linked through disulfide bonds at C₄₃₈ of SEQ ID NO:26 and C₇ of SEQ ID NO:27 to form an active Clostridial neurotoxin derivative. In some embodiments, the light chain derivative is at least 60%, 70%, 80%, 85%, 90%, 95%, 96%, 97%, 98%, or 99% identical to a SEQ ID NO:26. In some embodiments, the heavy chain derivative is at least 60%, 70%, 80%, 85%, 90%, 95%, 96%, 97%, 98%, or 99% identical to SEQ ID NO:27.

Since different types of signal peptides, tags, protease sites, and the orientation of these elements may vary in different embodiments, an exemplary sequence for a light chain derivative, according to the methods disclosed in the present application, is as follows in SEQ ID NO:28:

PFVNKQFNYK DPVNGVDIAY IKIPNAGQMQ PVKAFKIHNK
IWVIPERDTF TNPEEGDLNP PPEAKQVPVS YYDSTYLSTD
NEKDNYLKGV TKLFERIYST DLGRMLLTSI VRGIPFWGGS
TIDTELKVID TNCINVIQPD GSYRSEELNL VIIGPSADII
QFECKSFGHE VLNLTRNGYG STQYIRFSPD FTFGFEESLE
VDTNPLLGAG KFATDPAVTL AHQLIHAGHR LYGIAINPNR
VFKVNTNAYY EMSGLEVSFE ELRTFGGHDA KFIDSLQENE
FRLYYYNKFK DIASTLNKAK SIVGTTASLQ YMKNVFKEKY
LLSEDTSGKF SVDKLKFDKL YKMLTEIYTE DNFVKFFKVL
NRKTYLNFDK AVFKINIVPK VNYTIYDGFN LRNTNLAANF
NGQNTEINNM NFTKLKNFTG LFEFYKLLCV RGIITSHTQS
LDQGG

Since different types of signal peptides, tags, protease sites, and the orientation of these elements may vary in different embodiments, an exemplary minimal sequence for a heavy chain derivative, according to the methods disclosed in the present application, is as follows in SEQ ID NO:29:

ALNDLCIKVN NWDLFFSPSE DNFTNDLNKG EEITSDTNIE
AAEENISLDL IQQYYLTFNF DNEPENISIE NLSSDIIGQL
ELMPNIERFP NGKKYELDKY TMFHYLRAQE FEHGKSRIAL
TNSVNEALLN PSRVYTFFSS DYVKKVNKAT EAAMFLGWVE
QLVYDFTDET SEVSTTDKIA DITIIIPYIG PALNIGNMLY
KDDFVGALIF SGAVILLEFI PEIAIPVLGT FALVSYIANK
VLTVQTIDNA LSKRNEKWDE VYKYIVTNWL AKVNTQIDLI
RKKMKEALEN QAEATKAIIN YQYNQYTEEE KNNINFNIDD
LSSKLNESIN KAMININKFL NQCSVSYLMN SMIPYGVKRL
EDFDASLKDA LLKYIYDNRG TLIGQVDRLK DKVNNTLSTD
IPFQLSKYVD NQRLLSTFTE YINNIINTSI LNLRYESNHL
IDLSRYASKI NIGSKVNFDP IDKNQIQLFN LESSKIEVIL
KNAIVYNSMY ENFSTSFWIR IPKYFNSISL NNEYTIINCM
ENNSGWKVSL NYGEIIWTLQ DTQEIKQRVV FKYSQMINIS
DYINRWIFVT ITNNRLNNSK IYINGRLIDQ KPISNLGNIH
ASNNIMFKLD GCRDTHRYIW IKYFNLFDKE LNEKEIKDLY
DNQSNSGILK DFWGDYLQYD KPYYMLNLYD PNKYVDVNNV
GIRGYMYLKG PRGSVMTTNI YLNSSLYRGT KFIIKKYASG
NKDNIVRNND RVYINVVVKN KEYRLATNAS QAGVEKILSA
LEIPDVGNLS QVVVMKSKND QGITNKCKMN LQDNNGNDIG
FIGFHQFNNI AKLVASNWYN RQIERSSRTL GCSWEFIPVD
DGWGERPLGA G

The light chain and heavy chain derivatives of SEQ ID NO:28 and SEQ ID NO:29 are linked through disulfide bonds at C₄₂₉ of SEQ ID NO:28 and C₆ of SEQ ID NO:29 to form an active Clostridial neurotoxin derivative. In some embodiments, the light chain derivative is at least 60%, 70%, 80%, 85%, 90%, 95%, 96%, 97%, 98%, or 99% identical to a SEQ ID NO:28. In some embodiments, the heavy chain derivative is at least 60%, 70%, 80%, 85%, 90%, 95%, 96%, 97%, 98%, or 99% identical to SEQ ID NO:29.

In carrying out the methods described herein, contacting a subject with the Clostridium botulinum neurotoxins can be carried out by administering the isolated Clostridium botulinum neurotoxins to a subject inhalationally, parenterally, for example, subcutaneously, intravenously, intramuscularly, intraperitoneally, by intranasal instillation, or by application to mucous membranes, such as, that of the nose, throat, and bronchial tubes. The Clostridium botulinum neurotoxins may be administered alone or with suitable pharmaceutical carriers, and can be in solid or liquid form such as, tablets, capsules, powders, solutions, suspensions, or emulsions.

The neurotoxin derivative may also be administered parenterally. Solutions or suspensions can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that syringability is possible. It must be stable under the conditions of manufacture and storage and can be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), vegetable oils, hyaluronic acid, and suitable mixtures thereof.

Targeting the CNS may require intra-thecal or intra-ventricular administration. Administration may occur directly to the CNS. Alternatively, administration to the CNS may involve retrograde and transynaptic transport from peripheral neurons (motor neurons, nociceptors) to spinal ganglia (see Caleo et al., “A Reappraisal of the Central Effects of botulinum Neurotoxin Type A: By What Mechanism?” Journal of Neurochemistry 109:15-24 (2009), which is hereby incorporated by reference in its entirety).

Derivatives of a Clostridial neurotoxin of the present application can be used to augment the endogenous pharmaceutical activity of wild type Clostridial neurotoxins (e.g., BOTOX®), e.g., as a combination therapy.

Derivatives of a Clostridial neurotoxin can be administered as a conjugate with a pharmaceutically acceptable water-soluble polymer moiety. By way of example, a polyethylene glycol conjugate is useful to increase the half-life of the treatment compound, and to reduce the immunogenicity of the molecule. Specific PEG conjugates are described in U.S. Patent Application Publ. No. 2006/0074200 to Daugs et al., which is hereby incorporated by reference in its entirety. Other polymers used to form conjugates or mixtures include HA, which are described in U.S. Pat. No. 7,879,341 to Taylor and U.S. Patent Application Publication No. 2012/0141532 to Blanda et al., each of which is hereby incorporated by reference in its entirety. Liquid forms, including liposome-encapsulated formulations, are illustrated by injectable solutions and suspensions. Exemplary solid forms include controlled-release forms, such as a gel formulation, miniosmotic pump, or an implant. Other dosage forms can be devised by those skilled in the art, as shown, for example, by Ansel and Popovich, Pharmaceutical Dosage Forms and Drug Delivery Systems, 5^{th} Edition (Lea & Febiger 1990), Gennaro (ed.), Remington's Pharmaceutical Sciences, 19^{th} Edition (Mack Publishing Company 1995), and by Ranade and Hollinger, Drug Delivery Systems (CRC Press 1996), each of which is hereby incorporated by reference in its entirety.

According to one embodiment, by treatment it is meant dermatologic or aesthetic treatment (see e.g., Carruthers et al., “botulinum Toxin A in the Mid and Lower Face and Neck,” Dermatol. Clin. 22:151-158 (2004); Lang, “History and Uses of BOTOX® (botulinum Toxin Type A),” Lippincotts Case Manag. 9:109-112 (2004); Naumann et al., “Safety of botulinum Toxin Type A: A Systematic Review and Meta-Analysis,” Curr. Med. Res. Opin. 20:981-990 (2004); Vartanian et al., “Facial Rejuvenation Using botulinum Toxin A: Review and Updates,” Facial Plast. Surg. 20:11-19 (2004), which are hereby incorporated by reference in their entirety) as well as therapeutic treatment (see e.g., Bentsianov et al., “Noncosmetic Uses of botulinum Toxin,” Clin. Dermatol. 22:82-88 (2004); Carruthers et al., “Botox [BOTOX®]: Beyond Wrinkles,” Clin. Dermatol. 22:89-93 (2004); Jankovic, “botulinum Toxin In Clinical Practice,” J. Neurol. Neurosurg. Psychiatry 75:951-957 (2004); Klein, “The Therapeutic Potential of botulinum Toxin,” Dermatol. Surg. 30:452-455 (2004); Schurch, “The Role of botulinum Toxin in Neurology,” Drugs Today (Banc) 40:205-212 (2004), which are hereby incorporated by reference in their entirety).

Subjects to be treated pursuant to the methods of the present application include, without limitation, human and non-human primates, or other animals such as dog, cat, horse, cow, goat, sheep, rabbit, or rodent (e.g., mouse or rat).

Preferred treatment methods of the present application include, but are not limited to, dermatologic or aesthetic treatment, gastroenterologic treatment, genitourinaric treatment, neurologic treatment, oncological treatment, and/or the treatment of any condition characterized by synaptopathology (see, e.g., Brose et al., “Synaptopathies: Dysfunction of Synaptic Function,” Biochem. Soc. Trans. 38:443-444 (2010); Yu & Lu, “Synapses and Dendritic Spines as Pathogenic Targets in Alzherimer's Disease,” Neural Plasticity 2012:1-8 (2012); Siskova et al., “Reactive Hypertrophy of Synaptic Varicosities Within the Hippocampus of Prion-Infected Mice,” Biochem Soc. Trans. 38:471-475 (2010); Warner et al., “TorsinA and DYT1 Dystonia: A Synaptopathy?” Biochem. Soc. Trans. 38:452-456 (2010); Rozas et al., “Presynaptic Dysfunction in Huntington's Disease,” Biochem Soc. Trans. 38:488-492 (2010); and Jones, “Errant Ensembles: Dysfunctional Neuronal Network Dynamics in Schizophrenia,” Biochem. Soc. Trans. 38:516-521 (2010), which are hereby incorporated by reference in their entirety). Treatment of a condition characterized by synaptopathology may involve the neuromodulation of the synapse by the neurotoxin derivative.

Dermatologic or aesthetic treatment includes, but is not limited to, treatment for Rhtyiddess (wrinkles) (Sadick et al., “Comparison of botulinum Toxins A and B in the Treatment of Facial Rhytides,” Dermatol. Clin. 22:221-226 (2004), which is hereby incorporated by reference in its entirety), including glabellar (Carruthers et al., “botulinum Toxin type A for the Treatment of Glabellar Rhytides,” Dermatol. Clin. 22:137-144 (2004); Ozsoy et al., “Two-Plane Injection of botulinum Exotoxin A in Glabellar Frown Lines,” Aesthetic Plast. Surg. 28:114-115 (2004); which are hereby incorporated by reference in their entirety), neck lines (Brandt et al., “botulinum Toxin for the Treatment of Neck Lines and Neck Bands,” Dermatol. Clin. 22:159-166 (2004), which is hereby incorporated by reference in its entirety), crow's feet (Levy et al., “botulinum Toxin A: A 9-Month Clinical and 3D In Vivo Profilometric Crow's Feet Wrinkle Formation Study,” J. Cosmet. Laser Ther. 6:16-20 (2004), which is hereby incorporated by reference in its entirety), and brow contour (Chen et al., “Altering Brow Contour with botulinum Toxin,” Facial Plast. Surg. Clin. North Am. 11:457-464 (2003), which is hereby incorporated by reference in its entirety). Other dermatologic treatment includes treatment for hypertrophic masseter muscles (Ahn et al., “botulinum Toxin for Masseter Reduction in Asian Patients,” Arch. Facial Plast. Surg. 6:188-191 (2004), which is hereby incorporated by reference in its entirety) and focal hyperhydrosis (Glogau, “Treatment of Hyperhidrosis with botulinum Toxin,” Dermatol. Clin. 22:177-185, vii (2004), which is hereby incorporated by reference in its entirety), including axillary (“botulinum Toxin (Botox [BOTOX®]) for Axillary Hyperhidrosis,” Med. Lett. Drugs Ther. 46:76 (2004), which is hereby incorporated by reference in its entirety) and genital (Lee et al., “A Case of Foul Genital Odor Treated with botulinum Toxin A,” Dermatol. Surg. 30:1233-1235 (2004), which is hereby incorporated by reference in its entirety).

Gastroentologic treatment includes, but is not limited to, treatment for esophageal motility disorders (Achem, “Treatment of Spastic Esophageal Motility Disorders,” Gastroenterol. Clin. North Am. 33:107-124 (2004), which is hereby incorporated by reference in its entirety), pharyngeal-esophageal spasm (Bayles et al., “Operative Prevention and Management of Voice-Limiting Pharyngoesophageal Spasm,” Otolaryngol. Clin. North Am. 37:547-558 (2004); Chao et al., “Management of Pharyngoesophageal Spasm with Botox [BOTOX®],” Otolaryngol. Clin. North Am. 37:559-566 (2004), which are hereby incorporated by reference in their entirety), and anal fissure (Brisinda et al., “botulinum Neurotoxin to Treat Chronic Anal Fissure: Results of a Randomized ‘Botox [BOTOX ] vs. Dysport [DYSPORT®]’ Controlled Trial,” Ailment Pharmacol. Ther. 19:695-701 (2004); Jost et al., “botulinum Toxin A in Anal Fissure: Why Does it Work?” Dis. Colon Rectum 47:257-258 (2004), which are hereby incorporated by reference in their entirety).

Genitourinaric treatment includes, but is not limited to, treatment for neurogenic dysfunction of the urinary tract (“Botulinic Toxin in Patients with Neurogenic Dysfunction of the Lower Urinary Tracts,” Urologia July-August:44-48 (2004); Giannantoni et al., “Intravesical Resiniferatoxin Versus botulinum-A Toxin Injections for Neurogenic Detrusor Overactivity: A Prospective Randomized Study,” J. Urol. 172:240-243 (2004); Reitz et al., “Intravesical Therapy Options for Neurogenic Detrusor Overactivity,” Spinal Cord 42:267-272 (2004), which are hereby incorporated by reference in their entirety), overactive bladder (Cruz, “Mechanisms Involved in New Therapies for Overactive Bladder,” Urology 63:65-73 (2004), which is hereby incorporated by reference in its entirety), and neuromodulation of urinary urge incontinence (Abrams, “The Role of Neuromodulation in the Management of Urinary Urge Incontinence,” BJU Int. 93:1116 (2004), which is hereby incorporated by reference in its entirety).

Neurologic treatment includes, but is not limited to, treatment for tourettes syndrome (Porta et al., “Treatment of Phonic Tics in Patients with Tourette's Syndrome Using botulinum Toxin Type A,” Neurol. Sci. 24:420-423 (2004), which is hereby incorporated by reference in its entirety) and focal muscle spasticity or dystonias (MacKinnon et al., “Corticospinal Excitability Accompanying Ballistic Wrist Movements in Primary Dystonia,” Mov. Disord. 19:273-284 (2004), which is hereby incorporated by reference in its entirety), including, but not limited to, treatment for cervical dystonia (Haussermann et al., “Long-Term Follow-Up of Cervical Dystonia Patients Treated with botulinum Toxin A,” Mov. Disord. 19:303-308 (2004), which is hereby incorporated by reference in its entirety), primary blepharospasm (Defazio et al., “Primary Blepharospasm: Diagnosis and Management,” Drugs 64:237-244 (2004), which is hereby incorporated by reference in its entirety), hemifacial spasm, post-stroke (Bakheit, “Optimising the Methods of Evaluation of the Effectiveness of botulinum Toxin Treatment of Post-Stroke Muscle Spasticity,” J. Neurol. Neurosurg. Psychiatry 75:665-666 (2004), which is hereby incorporated by reference in its entirety), spasmodic dysphonia (Bender et al., “Speech Intelligibility in Severe Adductor Spasmodic Dysphonia,” J. Speech Lang. Hear Res. 47:21-32 (2004), which is hereby incorporated by reference in its entirety), facial nerve disorders (Finn, “botulinum Toxin Type A: Fine-Tuning Treatment of Facial Nerve Injury,” J. Drugs Dermatol. 3:133-137 (2004), which is hereby incorporated by reference in its entirety), and Rasmussen syndrome (Lozsadi et al., “botulinum Toxin A Improves Involuntary Limb Movements in Rasmussen Syndrome,” Neurology 62:1233-1234 (2004), which is hereby incorporated by reference in its entirety). Other neurologic treatments include treatment for amputation pain (Kern et al., “Effects of botulinum Toxin Type B on Stump Pain and Involuntary Movements of the Stump,” Am. J. Phys. Med. Rehabil. 83:396-399 (2004), which is hereby incorporated by reference in its entirety), voice tremor (Adler et al., “botulinum Toxin Type A for Treating Voice Tremor,” Arch. Neurol. 61:1416-1420 (2004), which is hereby incorporated by reference in its entirety), crocodile tear syndrome (Kyrmizakis et al., “The Use of botulinum Toxin Type A in the Treatment of Frey and Crocodile Tears Syndrome,” J. Oral Maxillofac. Surg. 62:840-844 (2004), which is hereby incorporated by reference in its entirety), marginal mandibular nerve paralysis, pain control, and anti-nociceptive effects (Cui et al., “Subcutaneous Administration of botulinum Toxin A Reduces Formalin-Induced Pain,” Pain 107:125-133 (2004) and U.S. Patent Application Publication No. 2012/0064059 to Foster et al., which are hereby incorporated by reference in its entirety), including but not limited to pain after mastectomy (Layeeque et al., “botulinum Toxin Infiltration for Pain Control After Mastectomy and Expander Reconstruction,” Ann. Surg. 240:608-613 (2004), which is hereby incorporated by reference in its entirety) and chest pain of esophageal origin (Schumulson et al., “Current and Future Treatment of Chest Pain of Presumed Esophageal Origin,” Gastroenterol. Clin. North Am. 33:93-105 (2004), which is hereby incorporated by reference in its entirety). Another neurologic treatment amenable to the methods of the present application is headache (Blumenfeld et al., “botulinum Neurotoxin for the Treatment of Migraine and Other Primary Headache Disorders,” Dermatol. Clin. 22:167-175 (2004), which is hereby incorporated by reference in its entirety).

The methods of the present application are also suitable for treatment of cerebral palsy (Balkrishnan et al., “Longitudinal Examination of Health Outcomes Associated with botulinum Toxin Use in Children with Cerebral Palsy,” J. Surg. Orthop. Adv. 13:76-80 (2004); Berweck et al., “Use of botulinum Toxin in Pediatric Spasticity (Cerebral Palsy),” Mov. Disord. 19:S162-S167 (2004); Pidcock, “The Emerging Role of Therapeutic botulinum Toxin in the Treatment of Cerebral Palsy,” J. Pediatr. 145:S33-S35 (2004), which are hereby incorporated by reference in their entirety), hip adductor muscle dysfunction in multiple sclerosis (Wissel et al., “botulinum Toxin Treatment of Hip Adductor Spasticity in Multiple Sclerosis,” Wien Klin Wochesnchr 4:20-24 (2001), which is hereby incorporated by reference in its entirety), neurogenic pain and inflammation, including arthritis, iatrogenic parotid sialocele (Capaccio et al., “Diagnosis and Therapeutic Management of Iatrogenic Parotid Sialocele,” Ann. Otol. Rhinol. Laryngol. 113:562-564 (2004), which is hereby incorporated by reference in its entirety), and chronic TMJ pain and displacement (Aquilina et al., “Reduction of a Chronic Bilateral Temporomandibular Joint Dislocation with Intermaxillary Fixation and botulinum Toxin A,” Br. J. Oral Maxillofac. Surg. 42:272-273 (2004), which is hereby incorporated by reference in its entirety). Other conditions that can be treated by local controlled delivery of pharmaceutically active neurotoxin derivatives include intra-articular administration for the treatment of arthritic conditions (Mahowald et al., “Long Term Effects of Intra-Articular BoNT A for Refractory Joint Pain,” Annual Meeting of the American College of Rheumatology (2004), which is hereby incorporated by reference in its entirety), and local administration for the treatment of joint contracture (Russman et al., “Cerebral Palsy: A Rational Approach to a Treatment Protocol, and the Role of botulinum Toxin in Treatment,” Muscle Nerve Suppl. 6:S181-S193 (1997); Pucinelli et al., “Botulinic Toxin for the Rehabilitation of Osteoarthritis Fixed-Flexion Knee Deformity,” Annual Meeting of the Osteoarthitis Research Society International (2004), which are hereby incorporated by reference in their entirety). The methods of the present application are also suitable for the treatment of pain associated with various conditions characterized by the sensitization of nociceptors and their associated clinical syndromes, as described in Bach-Rojecky et al., “Antinociceptive Effect of botulinum Toxin Type A In Rat Model of Carrageenan and Capsaicin Induced Pain,” Croat. Med. 1 46:201-208 (2005); Aoki, “Evidence for Antinociceptive Activity of botulinum Toxin Type A in Pain Management,” Headache 43 Suppl 1:S9-15 (2003); Kramer et al., “botulinum Toxin A Reduces Neurogenic Flare But Has Almost No Effect on Pain and Hyperalgesia in Human Skin,” J Neurol. 250:188-193 (2003); Blersch et al., “botulinum Toxin A and the Cutaneous Nociception in Humans: A Prospective, Double-Blind, Placebo-Controlled, Randomized Study,” J Neurol. Sci. 205:59-63 (2002), which are hereby incorporated by reference in its entirety.

The neurotoxin derivatives may be customized to optimize therapeutic properties (See e.g., Chaddock et al., “Retargeted Clostridial Endopeptidases: Inhibition of Nociceptive Neurotransmitter Release In Vitro, and Antinociceptive Activity in In Vivo Models of Pain,” Mov. Disord. 8:S42-S47 (2004); Finn, “botulinum Toxin Type A: Fine-Tuning Treatment of Facial Nerve Injury,” J. Drugs Dermatol. 3:133-137 (2004); Eleopra et al., “Different Types of botulinum Toxin in Humans,” Mov. Disord. 8:S53-S59 (2004); Flynn, “Myobloc,” Dermatol. Clin. 22:207-211 (2004); and Sampaio et al., “Clinical Comparability of Marketed Formulations of botulinum Toxin,” Mov. Disord. 8:S129-S136 (2004), which are hereby incorporated by reference in their entirety).

The derivative of a Clostridial neurotoxin may also be used, pursuant to the treatment methods of the present application, to treat diseases influenced by activity-dependent changes in synaptic structure (e.g., synaptopathologies) or hyperactivity of synapse forming apparatus (e.g., tubulin polymerization), and conditions associated with the proliferation of microtubules. For example, Alzheimer's Disease, Parkinson's Disease, and neuronal cancers (of both neural and glial origin). Other conditions that may be treated by the method of the present application include conditions where the synaptic complex is a disease target.

In one embodiment, neurotoxin derivatives of the present application accumulate within neuronal cytosol in higher amounts than wild-type Clostridial neurotoxin. In another embodiment, neurotoxin derivatives of the present application accumulate in muscle tissue in higher amounts than wild-type Clostridial neurotoxin. In some embodiments, neurotoxin derivatives of the present application are translocated at higher amounts than wild-type Clostridial neurotoxin.

### EXAMPLES

### Example 1—In-Vivo Pharmaceutical Activity Experiments for BoNT A/ad-0

Material and Methods

An atoxic (or reduced toxicity) derivative of Clostridium botulinum serotype A (“BoNT A/ad”), as described in U.S. Pat. No. 7,785,606 to Ichtchenko and Band (which is hereby incorporated by reference in its entirety), was expressed as described. Since this neurotoxin derivative is atoxic and does not possess a cargo attachment peptide sequence at its N-terminus, it was designated “BoNT A/ad-0,” where “ad-0” means atoxic derivative with no cargo site (0), as described herein. BoNT A/ad-0 was purified to electrophoretic homogeneity and activated by specific protease cleavage as described in Band et al., “Recombinant Derivatives of botulinum Neurotoxin A Engingeered for Trafficking Studies and Neuronal Delivery,” Protein Expression & Purification 71:62 (2010), which is hereby incorporated by reference in its entirety. The purified protein was prepared as a stock at a concentration of 10 mg/ml in PBS containing 40% glycerol for stabilization. The studies described below, evaluate the recombinant molecule's toxicity and pharmacologic activity.

Animals

Mice: female Balb/C mice, 5 to 7 weeks old; weight around 19 +/−3 grams.

Digit Abduction Score (DAS) Assay

A modification of the classic mouse Digit Abduction Scoring (“DAS”) assay was used to determine local pharmacologic activity in muscle, measured by muscle weakening effectiveness, as described in Aoki, “Preclinical Update on BOTOX® (botulinum Toxin Type A)-Purified Neurotoxin Complex Relative to Other botulinum Neurotoxin Preparations,” European Journal of Neurology (1999), which is hereby incorporated by reference in its entirety. In the DAS Assay, mice are suspended by their tails briefly to elicit a characteristic startle response in which the animal extends its hind limbs and abducts its hind digits. The DAS assay is especially useful to compare the muscle weakening effectiveness of different BoNT preparations (Aoki, “Preclinical Update on BOTOX® (botulinum Toxin Type A)-Purified Neurotoxin Complex Relative to Other botulinum Neurotoxin Preparations,” European Journal of Neurology (1999) and Aoki, “A Comparison of the Safety Margins of botulinum Neurotoxin Serotypes A, B, and F In Mice,” Toxicon 39:1815-1820 (2001), which are hereby incorporated by reference in their entirety).

This test was utilized to define pharmacological activity of BoNT A/ad-0 in mice. Mice were scored as having a positive DAS response when they were unable to fully extend all digits on the injected leg. A negative score is given to mice that spread the toes of the injected leg comparable to that of the non-injected leg.

Female Balb/C mice were given unilateral gastrocnemius intramuscular injections with the concentration described in a volume of 3 μl of 0.9% NaCl using a 25 μl Hamilton syringe. Muscle weakness was assessed from day 1 until 5 days post injection by suspending the mice in order to elicit a characteristic startle response and observing whether the toes on the injected leg were spreading compared to the non injected leg.

Measuring Paralysis

Definitive paralysis is described using two independent variables. First, the inability to use the injected leg to walk (paralysis); and second, the inability to spread the toes on the injected leg (digital abduction).

Results: Toxicity, LD₅₀

The BoNT A/ad-0 preparation described above was used for the following toxicity study. The study was designed to approximate the standard murine LD₅₀ test for wild type BoNT A (“wt BoNT A”). See Pearce et al., “Measurement of botulinum Toxin Activity: Evaluation of the Lethality Assay,” Toxicol Appl Pharmacol. 128:69-77 (1994), which is hereby incorporated by reference in its entirety).

A total of 30 female mice were used in this study. Each mouse was injected intraperitoneally with the indicated dose of BoNT A/ad-0 in 200 μl of PBS (Table 1), and observed for 24 hours.

Doses ranging from 0.5 pg/mouse to 2 μg/mouse, based on the LD₅₀ published by Pellett et al., “Neuronal Targeting, Internalization, and Biological Activity of a Recombinant Atoxic Derivative of botulinum Neurotoxin A,” Biochemical & Biophysical Research Communications 405(4):673-677 (2011), which is hereby incorporated by reference in its entirety), using BoNT A/ad (1.2 μg per mouse or 50 μg/kg body weight. The LD₅₀ for BoNT A/ad-0 was found to be very similar to that for BoNT A/ad (Table 1). Briefly, 50% or 5 out of 10 mice injected with a dose of 50 μg/kg body weight showed symptoms of botulism intoxication by 36 hours. All mice injected with a dose of 2μg, which is approximately 83.3 μg/kg body weight, expired within 48 hours. From this study it is concluded that 50 μg/kg body weight is the approximate LD₅₀ of BoNT A/ad-0.

TABLE 1
Results of Toxicity (LD50) Study for BoNT A/ad-0

| Injected Dose | No. Mice | Dead | Survive |
|---|---|---|---|
| 2 μg | 10 | 10 | 0 |
| 1.2 μg | 10 | 5 | 5 |
| 1 μg | 5 | 1 | 4 |
| 0.5 μg | 5 | 0 | 5 |

The LD₅₀ of wt BoNT A is approximately 0.5 ng/kg (Aoki, “A Comparison of the Safety Margins of botulinum Neurotoxin Serotypes A, B, and F In Mice,” Toxicon 39:1815-1820 (2001), which is hereby incorporated by reference in its entirety), or 100,000-fold lower than that of BoNT A/ad-0. Because of this toxicity, the effectiveness of wt BoNT A at extremely low doses, and the variability in potency for BoNTs produced from a wild type bacterial source, pharmacological doses of wt BoNT A are generally specified in terms of “activity units,” with 1 mouse LD₅₀ of wt BoNT A considered to be 1 activity unit, or approximately 0.5 ng/kg of wt BoNT A (Aoki, “A Comparison of the Safety Margins of botulinum Neurotoxin Serotypes A, B, and F In Mice,” Toxicon 39:1815-1820 (2001), which is hereby incorporated by reference in its entirety). This takes into account concentration variations in the level of active toxin between preparations and manufacturers. Harmonized standards across producers remain undefined. This is due to both different manufacturing methods, batch-to-batch variation and difference in the mouse leathality assay protocol across different manufacturers, but is also related to marketing claims. The final pharmaceutical preparations are formulated with albumin (BOTOX®) and/or lactose (DYSPORT®)). From the LD₅₀ results described here, it can be concluded that 1 LD₅₀ Unit (1U) of BoNT A/ad-0 corresponds to a dose of approximately 50 μg/kg, or approximately 1.2 μg per mouse.

Results: Muscle Paralysis Study/DAS Assay for Pharmacologic Activity In Vivo

BoNT A/ad-0 described above was tested in the murine DAS to determine if BoNT A/ad-0 possesses pharmacological activity at doses significantly below its LD₅₀, and whether it displays typical dose-response activity. Mice were injected in the gastrocnemius muscle with 3 μl of BoNT A/ad-0 in 0.9% NaCl using a 25 μl Hamilton Syringe. The doses administered are expressed as the μg administered per mouse, or units of BoNT A/ad-0 activity administered per mouse (Table 2).

Two observations are noted to categorize a mouse as positive for muscle paralysis induced by administration of BoNT A/ad-0. First, by the inability of the mouse to use the injected leg to walk (muscle paralysis). Second, by observing whether the digits on the injected leg appeared collapsed (digital abduction). Definite muscle paralysis was initially observed and recorded 24 hours after the initial administration. Mice were daily evaluated for definitive muscle paralysis for a maximum of 5 days.

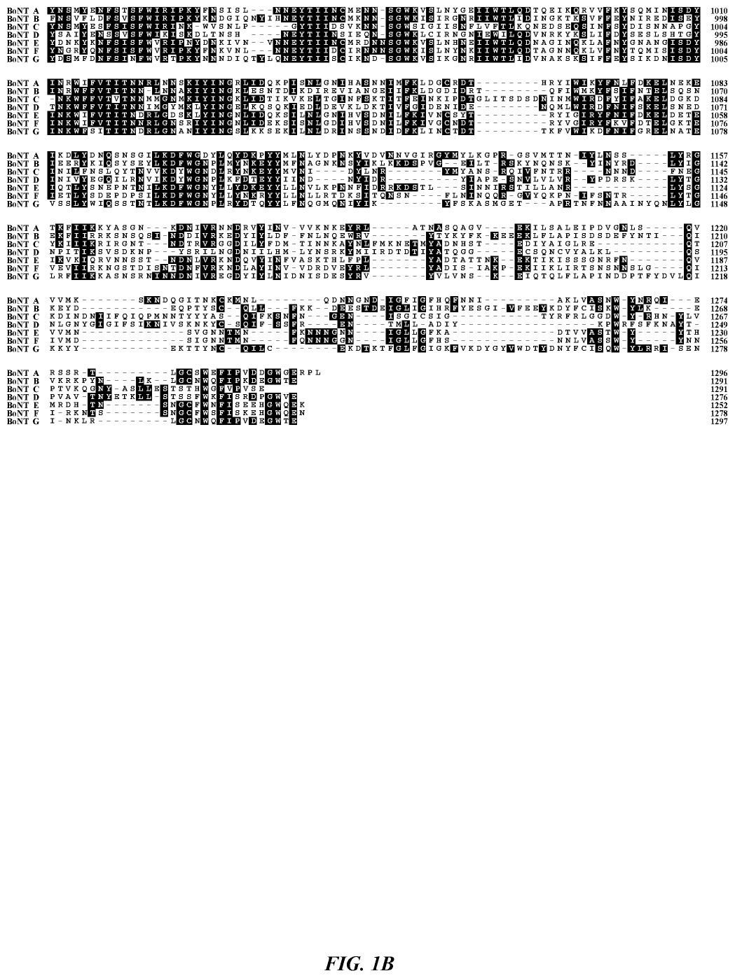

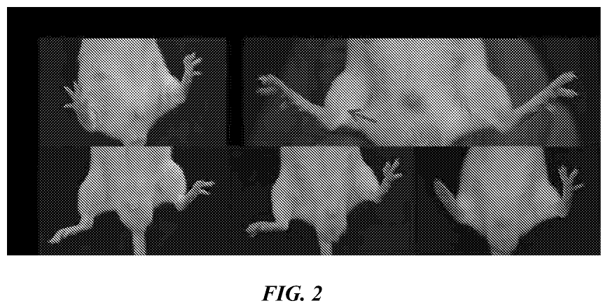

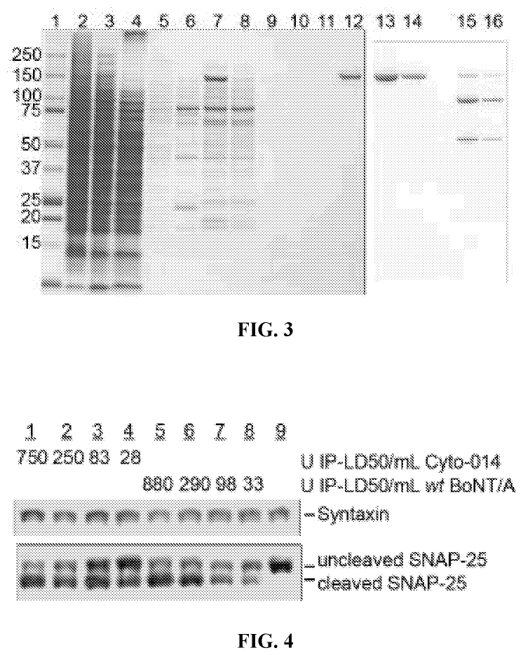

The results of this pharmacologic study of BoNT A/ad-0 are shown in Table 2 and FIG. 2. Mice administered doses ranging from 0.008 LD₅₀ units (0.01 μg) to 0.42 LD₅₀ units (0.5 μg) of BoNT A/ad-0 showed definitive muscle paralysis and digital abduction (FIG. 2 and Table 2), without any signs of mortality. In fact, 4 out of 5 animals injected with 0.01 μg presented with muscle paralysis and some degree of digital abduction (Table 2), indicating that the ED₅₀ for BoNT A/ad-0, the lowest dose at which 50% of the injected animals demonstrate the intended pharmacologic activity, is 0.01 μg or lower, which corresponds to 0.008 LD₅₀ units or lower. All mice that presented paralysis on day 1 continued to present paralysis to the end of the study, day 5. No signs of systemic toxicity were observed in any of the mice in this study.

These data confirm that BoNT A/ad-0 has similar pharmaceutical properties compared to wt BoNT A, albeit with a different dose-response profile, a significantly increased range of safe therapeutic activity and, therefore, an improved therapeutic index, and an improved safety margin. This comparison of BoNT A/ad-0 to pharmaceutical preparations of wt BoNT is illustrated in Table 3, and contrasted to the data reported by Aoki, “A Comparison of the Safety Margins of botulinum Neurotoxin Serotypes A, B, and F In Mice,” Toxicon 39:1815-1820 (2001), which is hereby incorporated by reference in its entirety. For instance, Aoki, “A Comparison of the Safety Margins of botulinum Neurotoxin Serotypes A, B, and F In Mice,” Toxicon 39:1815-1820 (2001), which is hereby incorporated by reference in its entirety, reported that the safety margin for BOTOX® is about 13.9 +/−1.7 and for DYSPORT® 7.6 +/−0.9. Here it is shown that at the lowest dose of BoNT A/ad-0 studied, 0.01 pg, definite paralysisis was observed in ⅘ mice. This dose can be considered a conservative estimate of the ED₅₀. Therefore, for BoNT A/ad-0, the safety margin is approximately 120, or expressed differently, approximately 10-fold better than that for BOTOX® or DYSPORT® (Table 3).

TABLE 2
Results of Pharmacologic Study of BoNT A/ad-0

| Dose | | | No. with | |
|---|---|---|---|---|
| Injected per | | | Definitive | No. |
| Mouse | LD₅₀ Units | No. Mice | Paralysis | Dead |
| 0 (placebo) | 0 | 9 | 0 | 0 |
| 0.01 μg | 0.008 | 5 | 4 | 0 |
| 0.1 μg | 0.08 | 5 | 5 | 0 |
| 0.5 μg | 0.42 | 10 | 10 | 0 |
| 1 μg | 0.83 | 5 | 5 | 0 |
| 1.2 μg | 1 | 5 | 2 | 3 |
| 1.5 μg | 1.25 | 5 | 1 | 4 |

Naïve mice were administered BoNT A/ad-0 in the left gastrocnemius via intramuscular injection with 3 μl containing the indicated mass or units of BoNT A/ad-0.

TABLE 3
LD50 and ED50 of BoNT A/ad-0

LD₅₀ = ~1.2 μg
ED₅₀ = ~0.01 μg (ED₅₀ = 0.01 μg or lower)
LD₅₀/ED₅₀ = safety margin = ~120

If expressed as units, the ED₅₀ of BoNT A/ad-0 is 0.008 LD₅₀ units, or lower.

Comparison to Prior Studies and Conclusions

Prior studies have found that mutations introduced into the light chain of recombinant BoNT A/ad (a molecule containing a cargo attachment peptide as described in U.S. Patent Application Publication No. 2011/0206616 to Ichtchenko and Band, which is hereby incorporated by reference in its entirety) increased the LD₅₀ of the toxin by 100,000-fold. However, experimentation determined that recombinant BoNT Cyto-012 can cause an immunogenetic response in mice. See Vasquez-Cintron et al., “Pre-Clinical Study of a Novel Recombinant botulinum Neurotoxin Derivative Engineered for Improved Safety,” Sci. Rep. 6:30429 (2016), which is hereby incorporated by reference in its entirety.

In the present study it was found that the LD₅₀ of BoNT A/ad-0, which has identical toxin-disabling mutations as BoNT A/ad, is likewise elevated ˜100,000-fold relative to wt BoNT A. But surprisingly, it was observed that BoNT A/ad-0 still possessed pharmacologic activity similar to that observed for wt BoNT A, and that a therapeutic agent need not be delivered via the cargo site of BoNT A ad to render it therapeutic. By comparing the dose-response of BoNT A/ad-0 to that reported for pharmaceutical preparations of wt BoNT A, it can be concluded that BoNT A/ad-0 can be used for pharmaceutical treatments in the same way as wt BoNTs, but with significantly reduced danger of systemic toxicity, and thus significant improved safety advantages for clinical use.

### Example 2—BoNT A Can Cause an Immunogenetic Response in Mice

As demonstrated in Example 1, the safety margin of botulinum neurotoxin (BoNT) can be improved by attenuating the activity of the light chain (LC) protease via substitution of amino acids in the protease active center. The molecules of Example 1 contain two amino acid substitutions (E224 >A and Y366 >A), and are approximately 100,000-fold less toxic than wt BoNT A1 (from which their sequence is derived), with a 2-fold improved safety margin (LD50/ED50 ratio).

However, experimentation determined that recombinant BoNTs of Example 1 can cause an immunogenetic response in mice. See Vasquez-Cintron et al., “Pre-Clinical Study of a Novel Recombinant botulinum Neurotoxin Derivative Engineered for Improved Safety,” Sci. Rep. 6:30429 (2016), which is hereby incorporated by reference in its entirety. To overcome this limitation, we developed a recombinant BoNT derivative which was only 10-fold less toxic than wild type BoNT A1 (“Cyto-014”), and evaluated its safety, effectiveness, and immunogenicity.

### Example 3—Cyto-014 Overcomes the Immunological Limitations of BoNT A

A recombinant BoNT derivative (“Cyto-014”, encoded by nucleotide sequence SEQ ID NO:18) with a single amino acid substitution in the metalloprotease domain was developed (Y366>F), in order to increase the potency of the derivative such that it was only 10-fold less toxic than wt BoNT A1, and which would thereby potentially overcome the immunological limitations of the molecules described in Example 1 and Vasquez-Cintron et al., “Pre-Clinical Study of a Novel Recombinant botulinum Neurotoxin Derivative Engineered for Improved Safety,” Sci. Rep. 6:30429 (2016), which is hereby incorporated by reference in its entirety. The molecular structure of Cyto-014 is identical to the previously tested Cyto-012 (the molecule described in Example 1), except that E224 is not substituted at all, and Y366 is replaced by phenylalanine (F) instead of alanine. This substitution is based on crystallographic studies of the BoNT A LC co-crystalized with its SNAP-25 substrate, and from studies of the catalytic activity of recombinant LC derivatives produced in E. coli. The substituting amino acid was chosen to be more structurally analogous to the amino acid substituted; thus instead of replacing Y366 with A, Y366 was replaced with F.

As described in more detail below, the resulting active BoNT derivative produced after processing (Cyto-014, as shown in SEQ ID NOs:21-24) was tested in vivo and in-vitro. In primary rat hippocampal cultures, Cyto-014 cleaved SNAP-25 under conditions similar to wt BoNT A1. The potency of Cyto-014 was determined by the standard murine lethality assay to have an IP-LD₅₀ 35 pg in 25 g mice, compared to 4.6 pg in a 25 g mice for wild type BoNT A1. Using the murine digital abduction assay, Cyto-014 was found to have an IM -ED₅₀ of 12 pg in 25 g mice compared to 0.6 pg in 25 g mice for wild type BoNT A1. The IM-LD₅₀ of Cyto-014 was 240 pg in 25 g mice compared to 6.22 pg in 25 g mice for wild type BoNT A1. Thus, Cyto-014 has a safety margin (IMLD₅₀/IMED₅₀ ratio) of 20 compared to a safety margin of 13 for wild type BoNT A1. When mice are subjected to repeat treatment 4 weeks after the first treatment with Cyto-014, overall treatment response (area under the DAS score curve) is similar to that for first injection. When the sera of mice receiving two injections of Cyto-014 was evaluated by ELISA for anti-BoNT A1 antibodies, there was no increase in immunoreactivity compared to mice receiving two injections of wt BoNT A1. When the same sera was evaluated for the presence of BoNT A neutralizing antibodies, no BoNT A neutralizing activity was found.

Expression

Cyto-014 was expressed and purified using previously published methods. FIG. 3 illustrates the expression, purification and activation of Cyto-014. Briefly, the Cyto-014 genetic construct is optimized for expression in insect cells. The construct includes an N-terminal signal sequence followed by a histidine affinity tag followed by the modified LC and heavy chain (HC) of BoNT A1, followed by strep affinity tags (SEQ ID NO:20). TEV protease cleavage sites have been placed between the affinity tags on the N- and C-termini, and between the LC and HC. The expressed proprotein is collected from the media (FIG. 3, lane 2, SEQ ID NO:20) as a soluble, stable proprotein, which is concentrated and purified by 2-step tandem affinity chromatography. The inactive proprotein is treated with TEV to enable simultaneous removal of the affinity tags and activation of the proprotein into the pharmacologically active disulfide-bonded heterodimer, and polished to remove impurities and produce the pure 150-kD Cyto-014 active drug (FIG. 3, lanes 15-16 reduced, SEQ ID NOs:21 (light chain) and 22 (heavy chain).

Cyto-014 IP-LD₅₀ Determination:

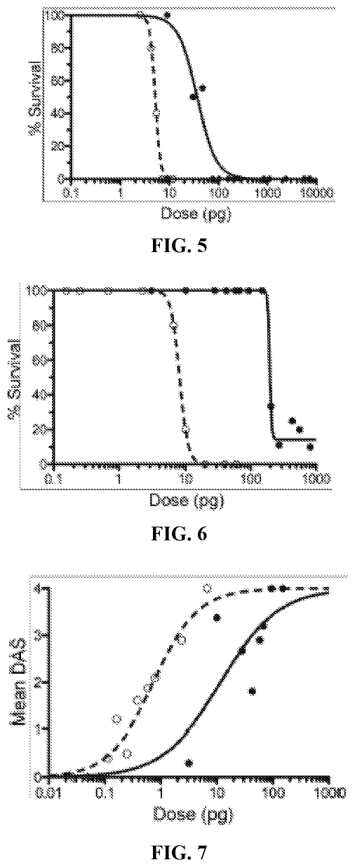

The mouse lethality assay (MLA) was used to determine the LD₅₀ of Cyto-014 and thereby define its Unit of activity (pg per LD₅₀ unit) in the fashion used to specify the activity of pharmaceutical BoNT products. Serial dilutions of purified Cyto-014 were injected into the intraperitoneal (IP) space of mice, and the average survival for each dosage group determined. A best-fit logistic regression curve calculated an IP-LD₅₀ (1 Unit activity) for Cyto-014 of 35 pg. This is ˜8-fold less toxic than wt BoNT A1 (FIG. 5 and Table 4).

Cyto-014 IM-ED50 and LD50 Determination:

A wide-ranged dose-response study was performed comparing Cyto-014 to wt BoNT A, using the DAS assay to quantify effectiveness and survival to quantify toxicity. Mice were injected into the gastrocnemius muscle, as previously described and paralysis was measured using the DAS assay. An evaluator blinded to treatment group performed DAS assessments. FIGS. 6 and 7 present the average survival and mean DAS response for each dosage group. The IM-LD₅₀ and IM-ED₅₀ were calculated from these curves by logistic regression analysis. The IM-LD₅₀ for Cyto-014 was determined to be 240 pg, and the IM-ED₅₀ 12 pg. This yields a calculated safety margin (IM-LD₅₀/ED₅₀ ratio) for Cyto-014 of 20.

TABLE 4
Summary of Safety Margin Results

| | IP-LD₅₀ | IM-LD₅₀ | IM-ED₅₀ | Safety Margin |
|---|---|---|---|---|
| | (95% Cl) | (95% Cl) | (95% Cl) | (95% Cl) |
| wt BoNT | 4.6 pg | 9.8 pg | 0.74 pg | 13 |
| A1 | (4.0-5.3) | (8.0-12) | (0.57-0.98) | (12-14) |
| Cyto-014 | 35 pg | 240 pg | 12 pg | 20 |
| | (22-56) | (190-300) | (6.8-19) | (16-28) |

The data described above show that Cyto-014 could be reliably produced in an Sf9-baculovirus system and purified to homogeneity. Cyto-014 was also found to have potency at the desired dose range, and to demonstrate an increased safety margin compared to wt BoNT A1. Cyto-014 was found to have an IMED₅₀ of 12 pg, combining the pharmacological activity of BoNT A1 with a lower risk of lethality. This IM-ED₅₀ is orders of magnitude lower than that reported for the previous double-mutant, Cyto-012, which should allow it to be repeatedly effective in mice without inducing a disadvantageous humoral immune response. These preliminary data suggest that Cyto-014 meets the criteria required for a rBoNT A derivative to have an improved safety margin, while remaining potent enough to minimize immunological reactivity and allow effective repeat treatment.

ELISA Studies

Enzyme-linked immunosorbent assays (ELISA) were carried out on 96-well plates as follows: 1) coating: with 4 μ.g/mL of Cyot-014 in 0.01M sodium bicarbonate buffer, pH 9.5, overnight; 2) blocking with 2% BSA, 200 μL/well, for two hours at room temperature; 3) incubating with sera diluted 1:50, 1:150, 1:450. 1:1250 in 0.1% BSA/PBS for 2 hours at room temperature; 4) incubating with 1:10,000 of 2Ab HRP-conjugate (anti-IgG and IgM); 5) develop with TMB solution for twenty minutes; and 6) stop reaction with H₂SO₄. Washes were performed after steps 1-4. This procedure was carried out separately on pooled and unpooled samples as follows.

First Bleed (pooled) ELISA protocol: small samples of serum (100 μL) were collected from the tail veins of mice two weeks after the first injection. Serum from individual mice per group (n=3-5) were pooled together to perform ELISA assay. Compromised serum samples (hemolytic, icteric, or lipemic) were omitted from the experiment. Samples were run in duplicates. All incubation step were performed at room temperature for one hour using 50 μL/well unless otherwise stated.

Second Bleed (unpooled) ELISA protocol: larger samples of serum were collected by cardiac puncture two weeks after the second injection. Serum from individual mice per group (n=3-5) were used without pooling to perform the ELISA assay. Samples were run in duplicates. All incubation step were performed at room temperature for one hour using 50 μL/well unless otherwise stated.

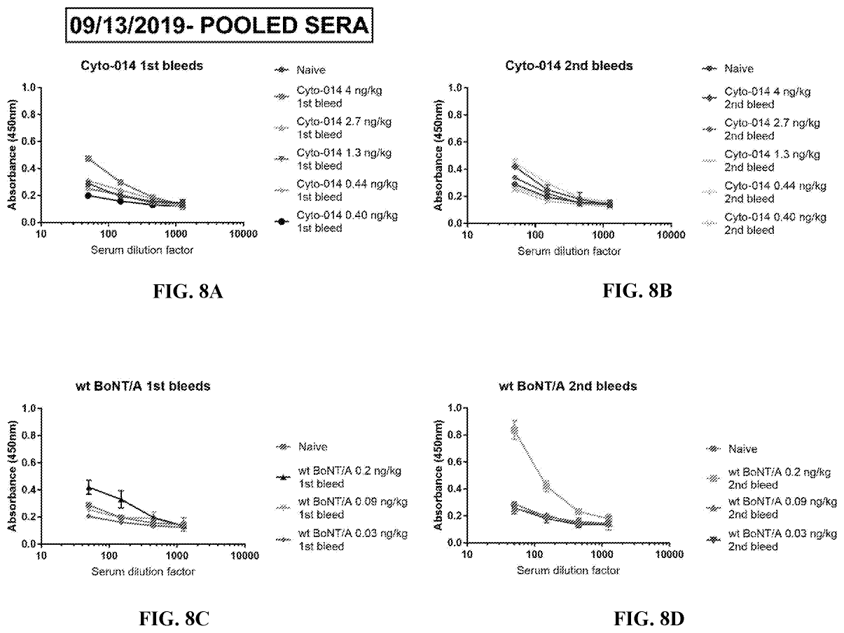

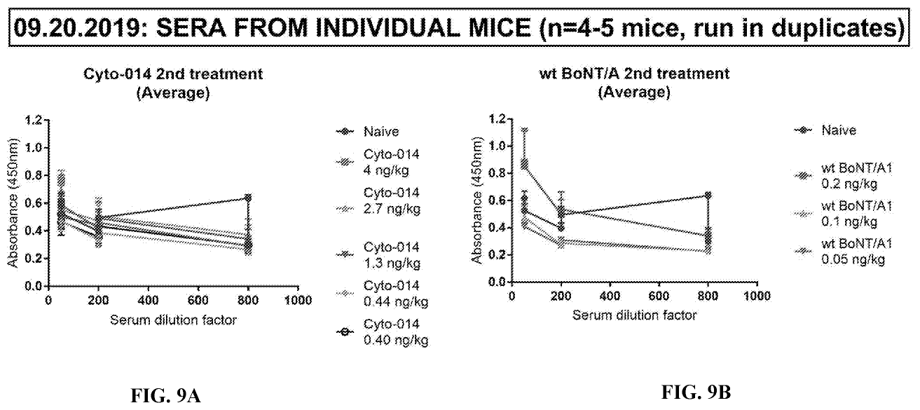

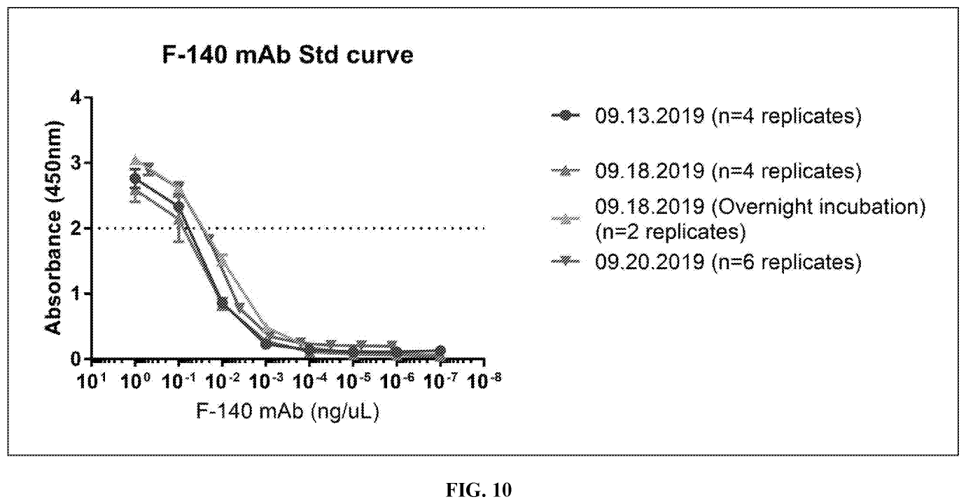

The results of the 2^{nd} bleed (pooled) ELISA experiments are shown in FIGS. 8A-8D. The results of the 2^{nd} bleed individual mouse sera (unpooled) ELISA are shown in FIGS. 9A-9B. The results for the positive control, using an antibody that recognizes BoNT A, are shown in FIG. 10. Thes results of these ELISA experiments demonstrate that when the sera of mice receiving two injections of Cyto-014 was evaluated by ELISA for anti-Cyto-014 antibodies, there was no increase in immunoreactivity compared to mice receiving two injections of wild type BoNT A1 despite administration at 10-fold higher doses.

Mouse Protection Assay

When the sera collected two weeks after the second injection was evaluated for the presence of BoNT A neutralizing antibodies using the mouse protection assay, no BoNT A neutralizing activity was found in the sera of the Cyto-014 treated mice. Wild type BoNT A (2IP-LD₅₀; ˜8 pg per mouse) ws mixed with mouse sera collected from animals injected with Cyto-014 or wild type BoNT A at a 6:1 toxin to neat serum ratio. The mixture was incubated for 30 minutes prior to injections. At time 0, mice were injected with 0.250 mL of toxin-serum mixture via the intraperitoneal cavity and monitored for 72 hours. Survival was used as a primary readout.

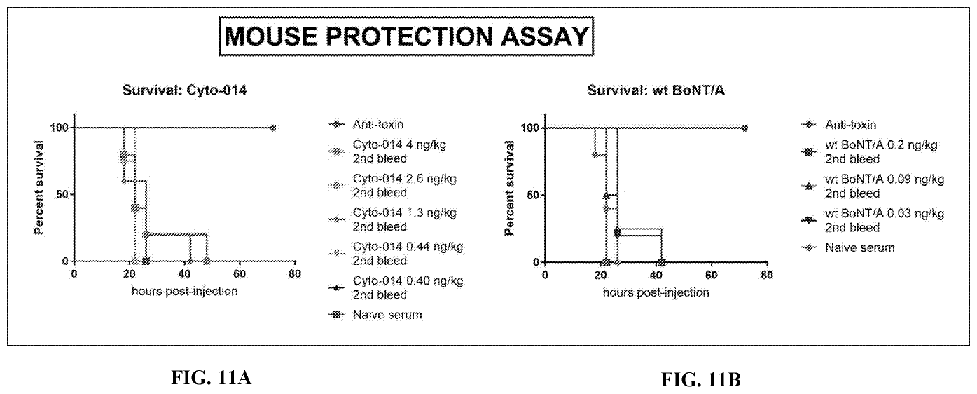

The results of the mouse protection assay are shown in FIGS. 11A-11B.

### Example 4—Cyto-013 Overcomes the Immunological Limitations of BoNT A

A recombinant BoNT derivative (“Cyto-013”), encoded by nucleotide sequence SEQ ID NO:19) with a single amino acid substitution in the metalloprotease domain is developed (E₂₂₄>Q) in order to increase the potency of the derivative such that it is only approximately 10-fold less toxic than wt BoNT A1, and which would thereby potentially overcome the immunological limitations of the molecules described in Example 1 and Vasquez-Cintron et al., “Pre-Clinical Study of a Novel Recombinant botulinum Neurotoxin Derivative Engineered for Improved Safety,” Sci. Rep. 6:30429 (2016), which is hereby incorporated by reference in its entirety. The molecular structure of Cyto-013 is identical to the previously tested Cyto-012 (the molecule described in Example 1), except that E₂₂₄ is substituted with a glutamine (Q) instead of an alanine, and Y₃₆₆ is not substituted at all. This substitution is based on crystallographic studies of the BoNT A LC co-crystalized with its SNAP-25 substrate, and from studies of the catalytic activity of recombinant LC derivatives produced in E. coli. The substituting amino acid was chosen to be more structurally analogous to the amino acid substituted; thus instead of replacing E₂₂₄ with A, E₂₂₄ is replaced with Q.

The immunological reactivity and efficacy of Cyto-013 is tested as described in Example 3 for Cyto-014.

Although the application has been described in detail for the purposes of illustration, it is understood that such detail is solely for that purpose, and variations can be made therein by those skilled in the art without departing from the spirit and scope of the application which is defined by the following claims.

What is claimed:

1. A recombinant Clostridium botulinum neurotoxin comprising:
a light chain of a Clostridium botulinum neurotoxin, wherein the light chain comprises a mutation corresponding to Y₃₆₆>X of BoNT A, wherein X is an amino acid that causes minimal structural interference to the light chain protease;
a heavy chain of a Clostridium botulinum neurotoxin, wherein the light and heavy chains are linked by a disulfide bond;
wherein the recombinant Clostridium botulinum neurotoxin has a 2-20 fold reduced toxicity compared to wild type Clostridium botulinum neurotoxin.

2. The recombinant Clostridium botulinum neurotoxin of claim 1, wherein X is phenylalanine.

3. The recombinant Clostridium botulinum neurotoxin of claim 1, wherein the recombinant Clostridium botulinum neurotoxin has a 5 fold reduced toxicity compared to wild type Clostridium botulinum neurotoxin.

4. A treatment method comprising:
selecting a subject in need of therapeutic treatment involving induction of muscle paralysis and contacting the subject with the recombinant Clostridium botulinum neurotoxin of claim 1 to induce muscle paralysis in the subject to treat the subject, with the proviso that the neurotoxin derivative does not possess a cargo attachment peptide sequence at its N-terminus.

5. The method according to claim 4, wherein the treatment is for a dermatologic or aesthetic condition selected from the group consisting of Rhytides, hypertrophic masseter muscles, and focal hyperhydrosis.

6. The method according to claim 4, wherein the treatment is for a gastroenterological condition selected from the group consisting of esophageal motility disorders, pharyngeal-esophageal spasm, and anal fissure.

7. The method according to claim 4, wherein the treatment is for a genitourinaric condition selected from the group consisting of neurogenic dysfunction of the urinary tract, overactive bladder, and neuromodulation of urinary urge incontinence.

8. The method according to claim 4, wherein the treatment is for a neurologic condition selected from the group consisting of tourettes syndrome, focal muscle spasticity or dystonias, cervical dystonia, primary blepharospasm, hemifacial spasm, spasmodic dysphonia, facial nerve disorders, Rasmussen syndrome, amputation pain, voice tremor, crocodile tear syndrome, marginal mandibular nerve paralysis, pain, chest pain of esophageal origin, headache, cerebral palsy, hip adductor muscle dysfunction in multiple sclerosis, neurogenic pain and inflammation, arthritis, iatrogenic parotid sialocele, and chronic TMJ pain and displacement.

9. A recombinant Clostridium botulinum neurotoxin comprising:
a light chain of a Clostridium botulinum neurotoxin, wherein the light chain comprises a mutation corresponding to E₂₂₄>X of BoNT A, wherein X is an amino acid that causes minimal structural interference to the light chain protease;
a heavy chain of a Clostridium botulinum neurotoxin, wherein the light and heavy chains are linked by a disulfide bond;
wherein the recombinant Clostridium botulinum neurotoxin has a 2-20 fold reduced toxicity compared to wild type Clostridium botulinum neurotoxin.

10. The recombinant Clostridium botulinum neurotoxin of claim 9, wherein X is glutamine.

11. The recombinant Clostridium botulinum neurotoxin of claim 9, wherein the recombinant Clostridium botulinum neurotoxin has a 5 fold reduced toxicity compared to wild type Clostridium botulinum neurotoxin.

12. A treatment method comprising:
selecting a subject in need of therapeutic treatment involving induction of muscle paralysis and
contacting the subject with the recombinant Clostridium botulinum neurotoxin of claim 9 to induce muscle paralysis in the subject to treat the subject, with the proviso that the neurotoxin derivative does not possess a cargo attachment peptide sequence at its N-terminus.

13. The method according to claim 12, wherein the treatment is for a dermatologic or aesthetic condition selected from the group consisting of Rhytides, hypertrophic masseter muscles, and focal hyperhydrosis.

14. The method according to claim 12, wherein the treatment is for a gastroenterological condition selected from the group consisting of esophageal motility disorders, pharyngeal-esophageal spasm, and anal fissure.

15. The method according to claim 12, wherein the treatment is for a genitourinaric condition selected from the group consisting of neurogenic dysfunction of the urinary tract, overactive bladder, and neuromodulation of urinary urge incontinence.

16. The method according to claim 12, wherein the treatment is for a neurologic condition selected from the group consisting of tourettes syndrome, focal muscle spasticity or dystonias, cervical dystonia, primary blepharospasm, hemifacial spasm, spasmodic dysphonia, facial nerve disorders, Rasmussen syndrome, amputation pain, voice tremor, crocodile tear syndrome, marginal mandibular nerve paralysis, pain, chest pain of esophageal origin, headache, cerebral palsy, hip adductor muscle dysfunction in multiple sclerosis, neurogenic pain and inflammation, arthritis, iatrogenic parotid sialocele, and chronic TMJ pain and displacement.
